(19) United States
(12) Patent Application Publication
(10) Pub. No.: US 2023/0285339 A1
(43) Pub. Date: Sep. 14, 2023

(54) THERAPIES FOR TREATING AML AND USES OF RARA AGONISTS, HYPOMETHYLATING AGENTS, AND BCL-2 INHIBITORS

(71) Applicant: Syros Pharmaceuticals, Inc., Cambridge, MA (US)

(72) Inventors: Qing Kang-Fortner, Framingham, MA (US); Christopher M. Fiore, Somerville, MA (US)

(21) Appl. No.: 18/019,981

(22) PCT Filed: Aug. 6, 2021
(86) PCT No.: PCT/US2021/045087

§ 371 Date: Feb. 6, 2023

Related U.S. Application Data
(60) Provisional application No. 63/121,760, filed on Dec. 4, 2020.
(60) Provisional application No. 63/115,541, filed on Nov. 18, 2020.
(60) Provisional application No. 63/062,350, filed on Aug. 6, 2020.

(51) Int. Cl. A61K 31/196; A61P 35/02

(52) U.S. Cl. CPC A61K 31/196; A61P 35/02

(57) ABSTRACT

The present disclosure features, inter alia, methods of treating a patient who has been diagnosed with acute myelomonocytic leukemia (the M4 subtype of AML), acute monocytic leukemia (the M5 subtype of AML), or myelodysplastic syndrome (MDS). The methods include administering to the patient a therapeutically effective amount of a retinoic acid receptor-alpha (RARA) agonist or a pharmaceutically acceptable salt thereof. In one or more embodiments (e.g., in treating MDS), administering the RARA agonist or the pharmaceutically acceptable salt thereof commences prior to determining whether the patient expresses a RARA biomarker and/or without consideration of the status of the RARA biomarker.

### CROSS-REFERENCE TO RELATED APPLICATIONS

This application claims the benefit of the filing date of U.S. Provisional Application No. 63/062,350, filed Aug. 6, 2020; of U.S. Provisional Application No. 63/115,541, filed Nov. 18, 2020; and of U.S. Provisional Application No. 63/121,760, filed Dec. 4, 2020. The entire contents of each of the foregoing provisional applications are hereby incorporated herein by reference in their entireties.

### BACKGROUND

Annually, nearly 200,000 people are diagnosed with a leukemia, lymphoma, or myeloma in the United States alone. One such cancer, acute myeloid leukemia (AML) affects both the bone marrow and blood. It can result from prior therapy (e.g., exposure to topoisomerases II, alkylating agents, or radiation) or from an underlying hematological disorder (e.g., myelodysplastic syndrome (MDS)). However, in many instances, it appears suddenly in previously healthy individuals. The pathogenesis of AML at the genetic level is heterogeneous. Genetic alterations observed in AML include an internal tandem duplication in a tyrosine kinase gene, chromosomal rearrangements that alter the functioning of genes involved in leukemogenesis, mutations resulting in activation of transcription factors, and others. Treatments developed for AML include cytotoxic chemotherapies, immunotherapies, hypomethylating agents (HMAs), and targeted small molecule therapies. Not all these options are available for every patient; some patients are deemed ineligible due to their age, performance status, or co-morbid conditions. Other patients are resistant to treatment, in which case their cancers are refractory to treatment or relapse occurs quickly. There is a critical need for therapeutic strategies that decrease this risk of relapse and improve the survival of patients with AML, other leukemias, lymphomas, and myelomas.

### SUMMARY

The present invention features, inter alia, methods of treating a patient (e.g., an adult or pediatric patient) who has been diagnosed with a type or subtype of cancer described herein (e.g., a subtype of acute myeloid leukemia (AML; e.g., the M4 or M5 subtype), myelodysplastic syndrome (MDS), chronic myelomonocytic leukemia (CMML), chronic lymphocytic leukemia (CLL (e.g., with 17p deletion)), acute lymphoblastic leukemia (ALL), small lymphocytic lymphoma (SLL), multiple myeloma (MM), non-Hodgkin lymphoma (NHL), and mantle cell lymphoma (MCL) with a RARA (retinoic acid receptor-alpha) agonist or a pharmaceutically acceptable salt thereof (e.g., tamibarotene or a pharmaceutically acceptable salt thereof) or a combination of the therapeutic agents described herein (e.g., a combination of one or more of a RARA agonist (e.g., the RARA-selective agonist tamibarotene), a hypomethylating agent (e.g., azacitidine or decitabine), and a Bcl-2 inhibitor (e.g., venetoclax)) either in the event the patient is newly diagnosed (ND) with the cancer, in which case the diagnosis may include a determination that the patient's cancer expresses one or more biomarkers (e.g., a biomarker of the monocytic expression signature (MES), or a correlate thereof, as described herein, alone or in combination with a RARA biomarker) or in the event the patient is resistant to treatment (e.g., is relapsed or refractory (R/R) to treatment) with venetoclax. These methods constitute a first specific embodiment of the invention, and in the methods of the first embodiment, a patient having a cancer as specified can be treated with the agent(s) specified prior to determining whether cancer cells in a biological sample obtained from the patient express a RARA biomarker (as described below and, for example, in U.S. Pat. No. 9,845,508, the content of which is hereby incorporated by reference herein in its entirety). In the methods of the first embodiment, a patient having a cancer as specified can be treated with the agent(s) specified without consideration of the status of the RARA biomarker. In the methods of the first embodiment, a patient having a cancer as specified can be treated with the agent(s) specified after determining cancer cells in a biological sample obtained from the patient express at least one biomarker indicative of a monocytic phenotype (e.g., a monocytic expression signature (MES)). In the methods of the first embodiment, a patient having a cancer as specified can be treated with the agent(s) specified after determining cancer cells in a biological sample obtained from the patient express a RARA biomarker and at least one biomarker indicative of a monocytic phenotype (e.g., a MES).

The present invention features, inter alia, methods of treating a patient (e.g., an adult or pediatric patient) who has been diagnosed with a breast cancer (e.g., an estrogen receptor-positive and Bcl-2-positive metastatic breast cancer), or lung cancer (e.g., non-small cell lung cancer or small cell lung cancer (e.g., in which BCL2 expression is high relative to a referenced standard)) with a RARA (retinoic acid receptor-alpha) agonist or a pharmaceutically acceptable salt thereof (e.g., tamibarotene or a pharmaceutically acceptable salt thereof) or a combination of the therapeutic agents described herein (e.g., a combination of one or more of a RARA agonist (e.g., the RARA-selective agonist tamibarotene), a hypomethylating agent (e.g., azacitidine or decitabine), and a Bcl-2 inhibitor (e.g., venetoclax)) either in the event the patient is newly diagnosed (ND) with the cancer, in which case the diagnosis may include a determination that the patient's cancer expresses one or more biomarkers (e.g., a biomarker of the monocytic expression signature (MES), or a correlate thereof, as described herein, alone or in combination with a RARA biomarker) or in the event the patient is resistant to treatment (e.g., is relapsed or refractory (R/R) to treatment) with venetoclax. These methods constitute a second embodiment, and in the methods of the second embodiment, a patient having a cancer as specified can be treated with the agent(s) specified prior to determining whether cancer cells in a biological sample obtained from the patient express a RARA biomarker (as described below and, for example, in U.S. Pat. No. 9,845,508, the content of which is hereby incorporated by reference herein in its entirety). In the methods of the second embodiment, a patient having a cancer as specified can be treated with the agent(s) specified without consideration of the status of the RARA biomarker. In the methods of the second embodiment, a patient having a cancer as specified can be treated with the agent(s) specified after determining cancer cells in a biological sample obtained from the patient express at least one biomarker indicative of a monocytic phenotype (e.g., a monocytic expression signature (MES)). In the methods of the second embodiment, a patient having a cancer as specified can be treated with the agent(s) specified after determining cancer cells in a biological sample obtained from the patient express a RARA biomarker and at least one biomarker indicative of a monocytic phenotype (e.g., a MES). For ease of reading, we will not refer to both an agent and a pharmaceutically acceptable salt thereof at every opportunity. It is to be understood that where a given agent can be used, a pharmaceutically acceptable salt thereof that also exhibits therapeutic activity can also be used.

In some embodiments (e.g., where a patient is treated with tamibarotene alone, tami/aza or tami/aza/ven), the subtype of AML is acute myelomonocytic leukemia (the M4 subtype of AML), and the patient is an adult or pediatric patient and may be newly diagnosed (ND), deemed unfit for treatment with standard induction therapy (unfit), or resistant to treatment (e.g., relapsed from or refractory to treatment (R/R)). In other embodiments (e.g., where a patient is treated with tamibarotene alone, tami/aza or tami/aza/ven), the subtype of AML is acute monocytic leukemia (the M5 subtype of AML), and the patient is an adult or pediatric patient and may be ND, unfit, or resistant to treatment (e.g., R/R). In another embodiment, the cancer type is MDS, and the patient is an adult or pediatric patient. In other embodiments, the cancer type is ALL, CMML, CLL, SLL, MM, NHL, or MCL and the patient is an adult or pediatric patient and may be ND, unfit, or resistant to treatment (e.g., R/R). More specifically, an adult or pediatric patient as just described may be treated as described herein where the patient has demonstrated resistance to treatment with venetoclax (by, for example, failure to achieve a complete response (CR) or partial response (CRi), the patient has become refractory to treatment with venetoclax, or cancer cells within a biological sample from the patient have demonstrated resistance to venetoclax (e.g., in an ex vivo assay).

In other embodiments, the cancer type is a breast cancer (e.g., an estrogen receptor-positive and BCL2-positive metastatic breast cancer) or a lung cancer (e.g., a small cell lung cancer (e.g., in which BCL2 expression is high relative to a referenced standard)). The methods comprise administering to the patient a therapeutically effective amount of a RARA agonist (e.g., tamibarotene), as described further herein, or a pharmaceutically acceptable salt thereof. In any embodiment of the present methods, in patients resistant to treatment (e.g., R/R), administering the RARA agonist (e.g., a RARA-selective agonist such as tamibarotene) or the pharmaceutically acceptable salt thereof can commence prior to determining whether the patient expresses a RARA biomarker and/or without consideration of the status of the RARA biomarker (as described, for example, in U.S. Pat. No. 9,845,508, the content of which is hereby incorporated by reference herein in its entirety). The RARA biomarker can comprise elevated expression of a RARA RNA gene transcript (e.g., an enhancer RNA (eRNA), pre-mRNA, or mature mRNA) or a super enhancer associated with the RARA gene. As specified, a biological sample comprising cancer cells from a patient can be assessed for a biomarker or a combination thereof, either in addition to or instead of RARA, as described herein (e.g., a biomarker whose expression correlates with resistance to venetoclax; see, e.g., FIG. 2). For example, the biomarker can be or can comprise the expression level of a gene described herein as a part of a MES (e.g., the expression level of one or more of CD14, CLEC7A (CD369), CD86, CD68, LYZ, MAFB, CD34, ITGAM (CD11b), FCGR1A (CD64), RARA, and KIT (CD117) (e.g., the expression levels of KIT, CD64, CD86, and LYZ)), or a protein encoded thereby, and/or a correlate thereof (e.g., elevated expression of MCL1 and/or under expression of BCL2). For example, the biomarker can be the expression level of a combination of the genes CD34, KIT (CD117), and BCL2. One can assess the enhancer or super enhancer associated with a gene that contributes to the MES by, for example, the techniques described herein. In some embodiments, a patient may be tested for, or may have been determined to express, a RARA biomarker (as described, for example, in U.S. Pat. No. 9,845,508 or U.S. Pat. No. 9,868,994, both of which are hereby incorporated by reference herein in their entireties) in addition to another biomarker indicative of a MES and/or a correlate thereof (e.g., MCL1 and/or BCL2). For example, in the context of the present methods, a patient may have been determined to express a RARA biomarker and an MCL1 and/or BCL2 biomarker. In any embodiment, where a gene constituting a biomarker is driven by a super enhancer, one may assess the super enhancer in addition to or instead of the level of a gene transcript (e.g., an mRNA or a cDNA transcribed therefrom) (see U.S. Pat. Nos. 9,845,508 and 9,868,994). The ten genes specifically described herein as biomarkers of the monocytic phenotype were selected because they are commonly referenced monocyte and stem cell differentiation markers concordant between the various expression datasets used in our studies (i.e., TCGA, BEAT AML, and our own clinical trial data); other monocyte and stem cell differentiation markers and genes whose expression correlate therewith can be used in addition to, or instead of, any one or more of those specifically described herein.

In any embodiment of the present methods where a RARA agonist (e.g., tamibarotene) or a pharmaceutically acceptable salt thereof is administered, it can be administered alone or in combination with a therapeutically effective amount of a second therapeutic agent or therapeutically effective amounts of a plurality of additional therapeutic agents. The second therapeutic agent can be a hypomethylating agent (e.g., azacitidine or decitabine), a Bcl-2 inhibitor (e.g., venetoclax), or a combination thereof (e.g., the RARA agonist can be administered in combination with both a hypomethylating agent (e.g., azacitidine or decitabine) and a Bcl-2 inhibitor (e.g., venetoclax; e.g., tami/aza/ven). In other embodiments, and particularly where the treatment regimen includes venetoclax, one or more of the agents just listed can be administered together with low-dose cytarabine (LDAC; e.g., for the treatment of ND AML or a patient deemed unfit), obinutuzumab (e.g., for patients with CLL or SLL), rituximab (e.g., for patients with CLL or SLL, with or without 17p deletion), or an endocrine therapy (e.g., tamoxifen, for patients with ER-positive breast cancer).

In embodiments of the present methods, the patient can be newly diagnosed with: a subtype of AML (e.g., the M4 subtype of AML, the M5 subtype of AML, or non-APL AML), MDS, CMML, CLL (with or without 17p deletion), ALL, SLL, MM, NHL, MCL, a breast cancer (e.g., an estrogen receptor-positive and BCL2-positive metastatic breast cancer), or small cell lung cancer. The patient may be (or may have been) diagnosed with the M4 subtype of AML or the M5 subtype of AML by virtue of the French-American-British (FAB) classification system and/or by virtue of a gene or protein expression profile characteristic of the M4 subtype of AML or the M5 subtype of AML (e.g., an MES, as described herein or a biomarker that correlates therewith).

In any embodiment of the present methods, including the methods of the first embodiment, the RARA agonist can be as shown in FIG. 4 (e.g., the RARA agonist can be all-trans retinoic acid (ATRA) or tamibarotene).

Without limiting the invention to therapies (i.e., therapies comprising or consisting of the administration of tamibarotene) or combination therapies (i.e., therapies comprising or consisting of the administration of tamibarotene and an HMA (e.g., azacitidine or decitabine) and/or venetoclax) that provide patient benefit by way of any particular underlying mechanisms of action, the therapies and uses of tamibarotene described herein are expected to improve outcomes for patients (e.g., patients having AML or a subtype thereof (e.g., the M5 subtype)) who previously would have been treated with a Bcl-2 inhibitor alone (e.g., venetoclax) or with a Bcl-2 inhibitor and an HMA (e.g., azacitidine or decitabine). For example, the Applicant expects patients described herein (e.g., a patient with AML) who are treated with a RARA agonist, an HMA, and a Bcl-2 inhibitor (e.g., tami/aza/ven) to experience a better outcome than patients treated with only an HMA and a Bcl-2 inhibitor. More specifically, the Applicant expects patients treated with tamibarotene, azacitidine, and venetoclax to experience better outcomes than patients treated with azacitidine and venetoclax. The improved outcome can be manifest by any clinically meaningful measure, including overall response rate, overall survival, and the likelihood of a complete or partial response to treatment (i.e., a CR or CRi, respectively). It has been reported that approximately one-third of patients with newly diagnosed AML do not respond to venetoclax combined with an HMA, the present standard of care (DiNardo et al., Blood, 133(1):3-4, 2019; DiNardo et al., N. Engl. J. Med. 383:617-629, 2020), and AML patients with monocytic AML (the M4 or M5 subtype) are more resistant to treatment with venetoclax and azacitidine than AML patients with a more primitive form of the disease (FAB-M0/M1/M2; Pei et al., Cancer Discovery 10:536-551, 2020).

Herein, the Applicant describes therapeutic methods (i.e., methods of treating a patient), and it is to be understood that those methods may also be expressed and claimed in terms of a “use” of the administered therapeutic agent(s), and vice versa. For example, it is to be understood that where the Applicant describes a method of treating a patient who has been diagnosed with AML or MDS by administering to the patient therapeutically effective amounts of tamibarotene, azacitidine, and venetoclax, the Applicant is also describing the use of therapeutically effective amounts of tamibarotene, azacitidine, and venetoclax in treating a patient who has been diagnosed with acute myelogenous leukemia (AML) or MDS. More specifically, a limitation described herein in the context of a method of treatment (e.g., a particular combination of therapeutic agents or dosages thereof), is to be understood as a teaching of the corresponding use (i.e., the use of the particular combination of therapeutic agents or dosages thereof) in the treatment specified and vice versa.

### BRIEF DESCRIPTION OF THE DRAWINGS

FIGS. 1A-1C illustrate and concern the ability of an MES, emerging from analysis of biomarkers of monocytic or primitive AML, to define FAB status (M0, M1, M2, M4, M5) in patients from the TCGA dataset (FIG. 1A) and the Beat AML dataset (FIG. 1B). FIG. 1C is a Table summarizing the sensitivity of the analysis for each dataset (the proportion of correctly predicted monocytic samples to all truly monocytic samples (i.e., where there were X monocytic samples, Y of which were predicted to be monocytic by our MES, the sensitivity is Y/(X+Y))) and the specificity of the analysis for each dataset (i.e., the proportion of correctly predicted primitive samples to all truly primitive samples).

FIG. 2 illustrates a correlation of venetoclax sensitivity with the indicated expression features, as described in the examples below.

FIG. 3 illustrates a correlation of venetoclax sensitivity/resistance with all genes. CLEC7A, CD14, MAFB, LYZ, CD68, CD86, FCGR1A, RARA, ITGAM, MCL1, CD34, KIT, and BCL2 correlate as shown, and any one or more of these genes can be assessed in generating a gene expression profile for a patient diagnosed with MDS or AML to further diagnose and identify a subtype thereof (e.g., M0, M1, M2, M3, M4, or M5). In case of doubt, if desired, the proteins encoded thereby can be assessed alternatively or in addition to assessing gene expression levels.

FIG. 4 is a table illustrating the structures of RARA agonists useful in the methods described herein.

FIGS. 5A and 5B are graphs illustrating dose-response curves in monocytic- and primitive AML patient samples to venetoclax alone and venetoclax-plus-azacitidine (FIG. 5A) and RARA expression levels in these same subtypes of patient samples (FIG. 5B). Leukemic stem cells were isolated and treated ex vivo with venetoclax plus-or-minus azacitidine, and their RARA expression levels were normalized against the expression of all genes using the RNA-seq data (GEO GSE132511).

FIGS. 6A and 6B illustrate that high RARA expression identifies AML patient populations enriched for high monocytic gene expression in both the TCGA and Beat AML datasets (FIG. 6A, left- and right-hand graphs, respectively; see also the quantitation of FIG. 6B).

FIG. 7 is a pair of plots illustrating that, in primary AML cultures, RARA expression and MES are associated with resistance to venetoclax.

FIG. 8 is a series of plots in which RARA-positive and RARA-negative, ND unfit AML patients enrolled in a clinical trial with tamibarotene are assessed with regard to the MES, BCL2 levels (low), and MCL1 levels (high). The three plots to the left were generated from enrolled patients, and the three plots to the right were generated from enrolled patients who achieved CR/CRi upon treatment with tamibarotene-plus-azacitidine.

### DETAILED DESCRIPTION

The following definitions apply to the compositions, methods, and uses described herein unless the context clearly indicates otherwise. Moreover, the definitions apply to linguistic and grammatical variants of the defined terms (e.g., the singular and plural forms of a term), and some linguistic variants are particularly mentioned below (e.g., “administration” and “administering”).

The term “about,” when used in reference to a value, signifies any value or range of values that is plus-or-minus 10% of the stated value (e.g., within plus-or-minus 1%, 2%, 3%, 4%, 5%, 6%, 7%, 8%, 9% or 10%, inclusive, of the stated value). For example, a dose of about 10 mg means any dose as low as 10% less than 10 mg (9 mg), any dose as high as 10% more than 10 mg (11 mg), and any dose or dosage range therebetween (e.g., 9-11 mg; 9.1-10.9 mg; 9.2-10.8 mg; and so on). Where a stated value cannot be exceeded (e.g., 100%), “about” signifies any value or range of values that is up to and including 10% less than the stated value (e.g., a purity of about 100% means 90%-100% pure (e.g., 95%-100% pure, 96%-100% pure, 97%-100% pure etc . . . )). In the event an instrument or technique measuring a value has a margin of error greater than 10%, a given value will be about the same as a stated value when they are both within the margin of error for that instrument or technique. In case of doubt, the disclosure of “about” a certain amount is a disclosure of that amount (i.e., “about 10 mg” is a disclosure of 10 mg and a disclosure of “at least about 1.5-fold” is a disclosure of at least 1.5-fold).

The term “administration” and variants thereof, such as “administering,” refer to the administration of a compound described herein (e.g., a RARA agonist (e.g., tamibarotene), an HMA (e.g., azacitidine), a BCL2 inhibitor such as venetoclax, and pharmaceutically acceptable salts thereof) or a pharmaceutical composition containing one or more of such compounds to a subject (e.g., a human patient) or system (e.g., a cell- or tissue-based system that is maintained ex vivo); as a result of the administration, the compound or composition containing the compound is introduced to the subject (e.g., the patient) or system. In addition to active pharmaceutical ingredients, items used as positive controls, negative controls, and placebos, any of which can also be or include a compound, can also be “administered.” One of ordinary skill in the art will be aware of a variety of routes that can, in appropriate circumstances, be utilized for administration to a patient or system. For example, the route of administration can be oral (i.e., by swallowing a pharmaceutical composition) or may be parenteral. More specifically, the route of administration can be bronchial (e.g., by bronchial instillation), by mouth (i.e., oral), dermal (which may be or comprise topical application to the dermis or intradermal, interdermal, or transdermal administration), intragastric or enteral (i.e., directly to the stomach or intestine, respectively), intramedullary, intramuscular, intranasal, intraperitoneal, intrathecal, intratumoral, intravenous (or intra-arterial), intraventricular, by application to or injection into a specific organ (e.g., intrahepatic), mucosal (e.g., buccal, rectal, sublingual, or vaginal), subcutaneous, tracheal (e.g., by intratracheal instillation), or ocular (e.g., topical, subconjunctival, or intravitreal). Administration can involve intermittent dosing (i.e., doses separated by various times) and/or periodic dosing (i.e., doses separated by a common period of time (e.g., every so many hours, daily (e.g., once daily oral dosing), weekly, twice per week, etc.)). In other embodiments, administration may involve continuous dosing (e.g., perfusion) for a selected time (e.g., about 1-2 hours). Therapeutically effective amounts and dosing regimens are known in the art, and we expect such amounts and regimens can be used in the present methods, particularly where selected RARA agonists, azacitidine, decitabine, and venetoclax are employed.

The term “biological sample” refers to a sample obtained or derived from a biological source of interest (e.g., a tissue or organism (e.g., an animal or human patient) or cell culture). For example, a biological sample can be a sample obtained from an individual (e.g., a patient or an animal model) suffering from a disease (or, in the case of an animal model, a simulation of that disease in a human patient) to be diagnosed and/or treated by the methods of the present disclosure or from an individual serving in the capacity of a reference or control (or whose sample contributes to a reference or control population). The biological sample can contain a biological cell, tissue or fluid or any combination thereof. For example, a biological sample can be or can include ascites; blood; blood cells; a bodily fluid, any of which may include or exclude cells (e.g., tumor cells (e.g., circulating tumor cells (CTCs) found in at least blood or lymph vessels)); bone marrow or a component thereof (e.g., hematopoietic cells, marrow adipose tissue, or stromal cells); cerebrospinal fluid (CSF); feces; flexural fluid; free-floating nucleic acids (e.g., circulating tumor DNA); gynecological fluids; hair; immune infiltrates; lymph; peritoneal fluid; plasma; saliva; skin or a component part thereof (e.g., a hair follicle); sputum; surgically-obtained specimens; tissue scraped or swabbed from the skin or a mucus membrane (e.g., in the nose, mouth, or vagina); tissue or fine needle biopsy samples; urine; washings or lavages such as a ductal lavage or broncheoalveolar lavage; or other body fluids, tissues, secretions, and/or excretions. A biological sample may include cancer cells or immune cells, such as NK cells and/or macrophages, which are found in many tissues and organs, including the spleen and lymph nodes. Samples of, or samples obtained from, a bodily fluid (e.g., blood, CSF, lymph, plasma, or urine) may include tumor cells (e.g., CTCs) and/or free-floating or cell-free nucleic acids. Cells (e.g., cancer cells) within the sample may have been obtained from an individual patient for whom a treatment is intended. Samples used in the form in which they were obtained may be referred to as “primary” samples, and samples that have been further manipulated (e.g., by removing one or more components of the sample) may be referred to as “secondary” or “processed” samples. Such processed samples may contain or be enriched for a particular cell type, cellular component (e.g., a membrane fraction), or cellular material (e.g., one or more cellular proteins, DNA, or RNA (e.g., mRNA), which may be subjected to amplification).

The term “biologically active” describes an agent (e.g., a compound described herein) that produces an observable biological effect or result in a biological system or model thereof (e.g., in a human, other animal, or a system maintained in cell/tissue culture or in vitro). The “biological activity” of such an agent can manifest upon binding between the agent and a target (e.g., RARA or BCL2)), and it may result in modulation (e.g., induction, enhancement, or inhibition) of a biological pathway, event, or state (e.g., a disease state). For example, the agent can modulate a cellular activity (e.g., stimulation of an immune response or inhibition of homologous recombination repair), time spent in a phase of the cell cycle (which may alter the rate of cellular proliferation), or initiation of apoptosis or activation of another pathway leading to cell death (which may lead to tumor regression). A biological activity and, optionally, its extent, can be assessed using known methods to detect any given immediate or downstream product of the activity or any event associated with the activity (e.g., inhibition of cell growth or tumor regression).

The term “carrier” refers to a diluent, adjuvant, excipient, or other vehicle with which an active pharmaceutical agent (e.g., a compound of the present disclosure, or a pharmaceutically acceptable salt, solvate, stereoisomer, tautomer, or isotopic form thereof) is formulated for administration. The carrier, in the amount and manner incorporated into a pharmaceutical composition, will be non-toxic to the subject and will not destroy the biological activity of the active ingredient (e.g., the compound or other specified form thereof) with which it is formulated. The carrier can be a sterile or sterilizable liquid, such as a water (e.g., water for injection) or a natural or synthetic oil (e.g., a petroleum-based or mineral oil, an animal oil, or a vegetable oil (e.g., a peanut, soybean, sesame, or canola oil)). The carrier can also be a solid; a liquid that includes one or more solid components (e.g., a salt, for example, a “normal saline”); a mixture of solids; or a mixture of liquids.

The term “comparable” refers to two or more items (e.g., agents, entities, situations, sets of conditions, etc.) that are not identical to one another but are sufficiently similar to permit comparison therebetween so that one of ordinary skill in the art will appreciate that conclusions may reasonably be drawn based on differences or similarities observed. In some embodiments, comparable sets of conditions, circumstances, individuals (e.g., an individual patient or subject), or populations are characterized by a plurality of substantially identical features and one or a small number of varied features. One of ordinary skill in the art will understand, in context, what degree of identity is required in any given circumstance for two or more items to be considered comparable. For example, two items are comparable to one another when they have in common a sufficient number and type of substantially identical features to warrant a reasonable conclusion that any differences in results obtained or phenomena observed with the items are caused by or are indicative of the variation in those features that are varied. In some embodiments, a comparable item serves as a “control.” For example, a “control subject/population” can be an untreated (or placebo-treated) individual/population who/that is afflicted with the same disease as an individual/population being treated.

The term “combination therapy” refers to those situations in which a subject is exposed to two or more therapeutic regimens (e.g., two or more therapeutic agents (e.g., three agents)) to treat a single disease (e.g., a cancer as described herein). The two or more regimens/agents may be administered simultaneously or sequentially. When administered simultaneously, a dose of the first agent and a dose of the second agent are administered at about the same time, such that both agents exert an effect on the patient at the same time or, if the first agent is faster- or slower-acting than the second agent, during an overlapping period of time. When administered sequentially, the doses of the first and second agents are separated in time, such that they may or may not exert an effect on the patient at the same time. For example, the first and second agents may be given within the same hour or same day, in which case the first agent would likely still be active when the second is administered. Alternatively, a much longer period of time may elapse between administration of the first and second agents, such that the first agent is no longer active when the second is administered (e.g., all doses of a first regimen are administered prior to administration of any dose(s) of a second regimen by the same or a different route of administration, as may occur in treating a refractory cancer). For clarity, combination therapy does not require that individual agents be administered together in a single composition or at the same time, although in some embodiments, two or more agents, including a compound of the present disclosure and a second agent described herein, may be administered within the same period of time (e.g., within the same hour, day, week, or month). The term “determining,” when used in the context of determining whether cells (e.g., cancer cells (e.g., leukemic cells)) in a biological sample obtained from a patient express a biomarker indicative of a monocytic phenotype or a RARA biomarker, means learning whether the cells express the biomarker by, for example, conducting an assay or procuring the results of such an assay.

The terms “dosage form,” “formulation,” and “preparation” refer to compositions that contain a compound described herein (e.g., a RARA agonist, HMA, or BCL2 inhibitor), or to other biologically or therapeutically active ingredients suitable for use as described herein (e.g., in combination with a RARA agonist). The term “unit dosage form” refers to a physically discrete unit of or containing a compound described herein (e.g., a RARA agonist) or a pharmaceutically acceptable salt thereof. One or more of an additional/second agent can also be formulated, administered, or used as described herein in a unit dosage form. Each such unit can contain a predetermined quantity of the active pharmaceutical ingredient, which may be the amount prescribed for a single dose (i.e., an amount expected to correlate with a desired outcome when administered as part of a therapeutic or prophylactic regimen) or a fraction thereof (e.g., a unit dosage form (e.g., a tablet or capsule) may contain one half of the amount prescribed for a single dose, in which case a patient would take two unit dosage forms (i.e., two tablets or two capsules)). One of ordinary skill in the art will appreciate that the total amount of a composition or agent administered to a particular subject is determined by one or more attending physicians and may involve administration of multiple unit dosage forms (e.g., as described herein).

The term “dosing regimen” refers to the unit dosage form(s) administered to, or prescribed for, a patient, and typically includes more than one dose separated by periods of time (e.g., as described herein or known in the art). The dosage form(s) administered within a dosing regimen can be of the same unit dose amount or of different amounts. For example, a dosing regimen can include a first dose in a first dose amount, followed by one or more additional doses in a second dose amount that is the same as or different from the first dose amount.

An “effective amount” refers to an amount of an agent (e.g., a RARA agonist (e.g., tamibarotene or a pharmaceutically acceptable salt thereof)) or to the amounts of agents in a combination of agents (e.g., tami/aza or tami/aza/ven) that produce(s) the desired effect for which it is administered. In some embodiments, the term refers to an amount that is sufficient, when administered to a population suffering from or susceptible to a disease in accordance with a therapeutic dosing regimen, to treat the disease, in which case the effective amount may also be referred to as a “therapeutically effective amount.” One of ordinary skill in the art will appreciate that a therapeutically effective amount may not achieve a successful treatment in any particular individual (i.e., in any given individual patient). Rather, a therapeutically effective amount provides a desired pharmacological response in a significant or certain expected number of subjects when administered to a population of patients in need of such treatment. A reference to an effective amount may be a reference to an amount of an agent administered or an amount measured in one or more specific tissues (e.g., a tissue affected by the disease) or fluids (e.g., blood, saliva, urine, etc.) after administration.

An “elderly unfit” patient is a human patient at least 60 years of age who is determined by a physician to not be a candidate for standard induction therapy.

An “enhancer” is a region of genomic DNA that helps regulate the expression of a gene and which can do so when positioned far away from the gene (currently understood to be up to about 1 Mbp away). An enhancer may overlap, but is often not composed of, gene coding regions. An enhancer is often bound by transcription factors and designated by specific histone marks. “Enhancer RNA” (eRNA) is an RNA that includes RNA transcribed from the DNA of an enhancer.

“Improve(s),” “increase(s)” or “reduce(s)/decrease(s)” (and obvious variants thereof, such as “improved” or “improving”) are terms used to characterize the manner in which a value changes or has changed relative to a reference value. For example, a measurement obtained from a patient (or a biological sample obtained therefrom) prior to treatment can be increased or reduced/decreased relative to that measurement when obtained during or after treatment in the same patient, a control patient, on average in a patient population, or in biological sample(s) obtained therefrom. The value may be improved in either event, depending on whether an increase or decrease is associated with a positive therapeutic outcome.

A patient who is “newly diagnosed” is one who has not been previously diagnosed with a type or subtype of cancer as described herein (e.g., AML or MDS) and who is therefore unexposed to a first agent (i.e., a RARA agonist (e.g., tamibarotene)) or one or more second agents (i.e., an HMA (e.g., azacitidine) or Bcl-2 inhibitor (e.g., venetoclax)), in which case the patient may also be defined as treatment naïve.

A “pharmaceutical composition” or “pharmaceutically acceptable composition,” which we may also refer to as a “pharmaceutical formulation” or “pharmaceutically acceptable formulation,” is a composition/formulation in which an active agent (e.g., an active pharmaceutical ingredient (e.g., a RARA agonist or second agent as described herein)) is formulated together with one or more pharmaceutically acceptable carriers. The active agent/ingredient can be present in a unit dose amount appropriate for administration in a therapeutic regimen that shows a statistically significant probability of achieving a predetermined therapeutic effect when administered to a relevant population. The pharmaceutical composition may be specially formulated for administration in solid or liquid form, including such forms made for oral or parenteral administration. For oral administration, the pharmaceutical composition can be formulated, for example, as an aqueous or non-aqueous solution or suspension or as a tablet or capsule. For systemic absorption through the mouth, the composition can be formulated for buccal administration, sublingual administration, or as a paste for application to the tongue. For parenteral administration, the composition can be formulated, for example, as a sterile solution or suspension for subcutaneous, intramuscular, intravenous, intra-arterial, intraperitoneal, intra-tumoral, or epidural injection. Pharmaceutical compositions comprising an active agent/ingredient (e.g., a compound described herein or a specified form thereof) can also be formulated as sustained-release formulations or as a cream, ointment, controlled-release patch, or spray for topical application. Creams, ointments, foams, gels, and pastes can also be applied to mucus membranes lining the nose, mouth, vagina, and rectum. Any of the compounds described herein and any pharmaceutical composition containing such a compound may also be referred to as a “medicament.”

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, MALATle, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “patient” to which administration is contemplated as described herein includes, but is not limited to, humans, and the patient may be a male, female, transgendered or other-gendered person of any age group (e.g., a pediatric subject (e.g., infant, child, adolescent) or adult subject (e.g., young adult, middle-aged adult, or senior adult)). While the methods described herein are clearly intended for application and use with respect to human patients, including pediatric patients (e.g., a pediatric patient having the M4 or M5 subtype of AML or MDS), the present disclosure is not so limited, and other non-human animals can also be treated. That is, the present methods encompass veterinary applications.

The term “population” means a number of items (e.g., at least 30, 40, 50, or more) sufficient to reasonably reflect the distribution, in a larger group, of the value being measured in the population. Within the context of the present invention, the population can be a discrete number of humans, laboratory animals, cell lines, tissue samples, or a combination thereof that are identified by at least one common characteristic for the purposes of data collection and analysis. Cell lines and tissue samples are useful as such and when grown in a laboratory animal (e.g., a mouse) by implanting cells of the cell line or a tissue sample into the animal, which then give rise to a cell line-derived xenograft (CDX) or patient-derived xenograft (PDX). A “population of samples” refers to a plurality of samples that is large enough to reasonably reflect the distribution of a value (e.g., a value related to the state of a biomarker) in a larger group (e.g., a larger group of samples or patients). As noted, the entities within a population can have a common characteristic, rendering the population useful in setting a threshold level (i.e., the pre-determined threshold level discussed herein) or prevalence cutoff against which a value obtained from a patient who is within the larger group and has the same common characteristic can be assessed. For example, a population of samples containing cells that have, as a common characteristic, a feature of the M5 subtype of AML can be used to set a threshold level or prevalence cutoff for a biomarker (e.g., a RARA biomarker or monocytic biomarker) that can then be used to assess a sample of comparable cells from a patient diagnosed with the M5 subtype of AML.

The term “prevalence cutoff,” as used herein in reference to a specified value (e.g., the strength of a SE associated a biomarker disclosed herein) means the prevalence rank that defines the dividing line between two subsets of a population (e.g., a subset of “responders” and a subset of “non-responders,” which, as the names imply include patients who are likely or unlikely, respectively, to experience a beneficial response to a therapeutic agent or agents). Thus, a prevalence rank that is equal to or higher (e.g., a lower percentage value) than the prevalence cutoff defines one subset of the population; and a prevalence rank that is lower (e.g., a higher percentage value) than the prevalence cutoff defines the other subset of the population.

As used herein, the term “prevalence rank” for a specified value (e.g., the mRNA level of a specific biomarker) means the percentage of a population that are equal to or greater than that specific value. For example, a 35% prevalence rank for the amount of mRNA of a specific biomarker in a test cell means that 35% of the population have that level of biomarker mRNA or greater than the test cell.

The term “primary RNA transcript” as used herein refers to an RNA transcription product from a DNA sequence that includes a coding region of a gene (e.g., at least one exon) and/or a non-coding region of the gene (e.g., an intron or a regulatory region of the gene (e.g., an enhancer or super enhancer that regulates expression of the gene)). Thus, the primary RNA transcript can be an “enhancer RNA” or “eRNA” (when it includes RNA transcribed from the enhancer or super enhancer) a microRNA, a precursor mRNA (“pre-mRNA”) or mature mRNA. We may specify the source gene of a primary RNA transcript. For example, a “RARA primary RNA transcript” is a primary RNA transcript transcribed from the RARA gene, and a “biomarker indicative of a monocytic phenotype primary RNA transcript is a primary RNA transcript transcribed from a gene encoding a biomarker indicative of a monocytic phenotype (e.g., the gene CD14, CLEC7A (CD369), CD86, CD68, LYZ, MAFB, CD34, ITGAM (CD11b), FCGR1A (CD64), RARA, KIT (CD117); the gene MCL1; the gene CD34; the gene KIT (CD117); and the gene BCL2. In methods of assessing the level of expression of a primary RNA transcript, one may assess a cDNA that has been synthesized or reverse transcribed from a primary RNA transcript.

The term “rank” means a position assigned to an entity within a population based on a quantity associated with the entity and relative to the same quantity in other entities within the population. “Rank ordering” means the ordering of values from highest to lowest or from lowest to highest.

The term “RARA gene” refers to a genomic DNA sequence that encodes a functional retinoic acid receptor-α (RARA) and specifically excludes gene fusions that comprise all or a portion of the RARA gene. In some embodiments, the RARA gene is located at chr17:38458152-38516681 in genome build hg19.

The term “reference” describes a standard or control relative to which a comparison is made. For example, an agent, animal (e.g., a subject (e.g., an animal used in laboratory studies)), cell or cells, individual (e.g., an individual patient), population, sample (e.g., biological sample), sequence or value of interest is compared with a reference or control agent, animal (e.g., a subject (e.g., an animal used in laboratory studies)), cell or cells, individual (e.g., an individual patient), population, sample, or sequence or value, respectively. In some embodiments, a reference or control is tested and/or determined substantially simultaneously with the testing or determination of interest. In other embodiments, a reference or control is a historical reference or control, optionally embodied in a tangible medium. Typically, as would be understood by one of ordinary skill in the art, a reference or control is determined or characterized under comparable conditions to those under assessment, and one of ordinary skill in the art will appreciate when sufficient similarities are present to justify reliance on and/or comparison to a particular possible reference or control.

The term “response” with respect to a treatment may refer to any beneficial alteration in a patient's condition that occurs as a result of, or correlates with, treatment. Such an alteration may include stabilization of the condition (e.g., prevention of deterioration that would have taken place in the absence of the treatment (e.g., stable disease)), amelioration of symptoms of the condition, and/or improvement in the prospects for cure of the condition (e.g., tumor regression), etc. The response may be a cellular response (e.g., as assessed in a cancer cell) and can be measured using a wide variety of criteria, including clinical criteria and objective criteria, known in the art. Techniques for assessing a response include, but are not limited to, assay assessment, clinical examination, positron emission tomography, X-ray, CT scan, MM, ultrasound, endoscopy, laparoscopy, assessing the presence or level of tumor markers in a sample obtained from a subject, cytology, and/or histology. Regarding measuring tumor response, methods and guidelines for assessing response to treatment are discussed in Therasse et al. (J. Natl. Cancer Inst., 92(3):205-216, 2000). The exact response criteria can be selected by one of ordinary skill in the art in any appropriate manner, provided that when comparing groups of cancers and/or patients, the groups to be compared are assessed based on the same or comparable criteria for determining response rate.

As used herein, when the term “strength” is used to refer to a portion of an enhancer of a super enhancer, it means the area under the curve of the number of H3K27Ac or other genomic marker reads plotted against the length of the genomic DNA segment analyzed. Thus, “strength” is an integration of the signal resulting from measuring the mark at a given base pair over the span of the base pairs defining the region being chosen to measure.

As used herein, the term “super enhancer” or “SE” refers to a subset of enhancers that contain a disproportionate share of histone marks and/or transcriptional proteins relative to other enhancers in a particular cell or cell type. Genes regulated by SEs are predicted to be of high importance to the function of a cell. SEs are typically determined by rank ordering all of the enhancers in a cell based on strength and determining, using available software such as ROSE ((bitbucket.org/young_computation/rose), the subset of enhancers that have significantly higher strength than the median enhancer in the cell. As needed, one of ordinary skill in the art can consult, e.g., U.S. Pat. No. 9,181,580, which describes methods of identifying SEs that modulate the expression of cell type-specific genes (e.g., genes that define the identity of embryonic stem cells) and which is hereby incorporated by reference herein in its entirety.

The term “tami/aza” refers to a combination of tamibarotene or a pharmaceutically acceptable salt thereof and azacitidine or a pharmaceutically acceptable salt thereof.

The term “tami/aza/ven” refers to a combination of tamibarotene (or a pharmaceutically acceptable salt thereof), azacitidine (or a pharmaceutically acceptable salt thereof), and venetoclax (or a pharmaceutically acceptable salt thereof).

The terms “threshold” and “threshold level” mean a level that defines the dividing line between two subsets of a population (e.g., likely responders and non-responders). A threshold or threshold level can define a prevalence cutoff or a cutoff value and may be assessed with regard to various features of a biomarker (e.g., the level, ordinal rank, or prevalence rank of primary RNA transcripts expressed from the biomarker gene or the strength, ordinal rank, or prevalence rank of a super enhancer associated with the biomarker gene).

In one embodiment, the present invention features a method of treatment or the use of a therapeutically effective amount of tamibarotene or a pharmaceutically acceptable salt thereof in treating a patient who has been diagnosed with acute myelomonocytic leukemia (the M4 subtype of acute myeloid leukemia (AML)) or acute monocytic leukemia (the M5 subtype of AML); the tamibarotene or the pharmaceutically acceptable salt thereof can be administered to the patient (a) prior to determining whether leukemic cells in a biological sample obtained from the patient express a RARA biomarker and/or without consideration of the status of the RARA biomarker; (b) after determining leukemic cells in a biological sample obtained from the patient express at least one biomarker indicative of a monocytic phenotype; or (c) after determining leukemic cells in a biological sample obtained from the patient express a RARA biomarker and at least one biomarker indicative of a monocytic phenotype. This method/use constitutes a second specific embodiment of the invention.

In one embodiment, the present invention features a method of treatment or the use of a therapeutically effective amount of tamibarotene or a pharmaceutically acceptable salt thereof in treating a patient who has been diagnosed with myelodysplastic syndrome (MDS), wherein the tamibarotene or the pharmaceutically acceptable salt thereof is administered to the patient (a) prior to determining whether MDS cells in a biological sample obtained from the patient express a RARA biomarker and/or without consideration of the status of the RARA biomarker; (b) after determining MDS cells in a biological sample obtained from the patient express at least one biomarker indicative of a monocytic phenotype; or (c) after determining MDS cells in a biological sample obtained from the patient express a RARA biomarker and at least one biomarker indicative of a monocytic phenotype. This method/use constitutes a third specific embodiment of the invention.

In the second or third specific embodiments of the invention, (a) the RARA biomarker comprises (i) elevated expression, relative to a reference, of a RARA primary RNA transcript or a cDNA transcribed therefrom, or (ii) a super enhancer associated with the RARA gene and (b) the at least one biomarker indicative of a monocytic phenotype comprises (i) elevated expression, relative to a reference, of a primary RNA transcript from a CD14 gene, a CLEC7A (CD369) gene, a CD86 gene, a CD68 gene, a LYZ gene, an MAFB gene, a CD34 gene, an ITGAM (CD11b) gene, and/or an FCGR1A (CD64) gene, a cDNA transcribed therefrom, or a protein encoded thereby or (ii) a super enhancer associated with the CD14 gene, the CLEC7A (CD369) gene, the CD86 gene, the CD68 gene, the LYZ gene, the MAFB gene, the CD34 gene, the ITGAM (CD11b) gene, the FCGR1A (CD64) gene, a KIT (CD117) gene, the MCL1 gene and/or the BCL2. The tamibarotene or the pharmaceutically acceptable salt thereof can be administered in combination with a therapeutically effective amount of a second therapeutic agent or therapeutically effective amounts of a plurality of additional therapeutic agents. The second therapeutic agent can be a hypomethylating agent (e.g., azacitidine or decitabine). The second therapeutic agent can be a BCL2 inhibitor (e.g., venetoclax). The tamibarotene can be administered in combination with a hypomethylating agent and a BCL2 inhibitor, wherein the hypomethylating agent is azacitidine and the BCL2 inhibitor is venetoclax. The patient can one who has relapsed following treatment with venetoclax, the patient can be one who has become refractory to treatment with venetoclax, or leukemic cells or MDS cells within a biological sample obtained from the patient can demonstrate resistance to venetoclax. The patient can be newly diagnosed with the M4 subtype of AML, the M5 subtype of AML, or MDS and/or is considered unfit for standard induction chemotherapy. The patient can be one who has been diagnosed with the M4 subtype of AML, the M5 subtype of AML, or MDS by virtue of the French-American-British (FAB) classification system or by virtue of a gene or protein expression profile characteristic of the M4 subtype of AML, the M5 subtype of AML, or MDS. The at least one biomarker indicative of a monocytic phenotype can be a gene or protein having a level of expression, relative to a reference, that correlates with resistance to venetoclax, optionally wherein the at least one biomarker indicative of a monocytic phenotype comprises a gene selected from CD14, CLEC7A (CD369), CD86, CD68, LYZ, MAFB, CD34, ITGAM (CD11b), FCGR1A (CD64), or KIT (CD117), MCL1, and BCL2, a cDNA transcribed therefrom, or a protein encoded thereby. The plurality of additional therapeutic agents can consist of or comprise therapeutically effective amounts of azacitidine or decitabine, venetoclax, and low-dose cytarabine, in which case the patient can be one who is newly diagnosed with the M4 subtype of AML, the M5 subtype of AML, or MDS. The RARA biomarker and/or the at least one biomarker indicative of a monocytic phenotype can be assessed by determining whether cancer cells in a biological sample obtained from the patient have (a) a super enhancer associated with a RARA gene or a gene indicative of a monocytic phenotype, wherein the super enhancer has a strength or an ordinal rank based on its strength or prevalence that is equal to or above a pre-determined threshold level; and/or (b) a level of primary RNA transcript from the RARA gene or the gene indicative of a monocytic phenotype that is equal to or above a pre-determined threshold level. The RARA biomarker and the at least one biomarker indicative of a monocytic phenotype can consist of or comprise:
(a) a primary RNA transcript level from the RARA gene or a biomarker gene indicative of a monocytic phenotype, wherein the transcript level is elevated relative to a threshold level that defines a dividing line between patients who are likely to respond to tamibarotene and patients who are not likely to respond to tamibarotene and is pre-determined by analysis of primary RNA transcript levels in a population of samples comprising a cell line representing the M4 or M5 subtype of AML, a cell line representing MDS, a xenograft representing the M4 or M5 subtype of AML, a xenograft representing MDS, a biological sample from a patient suffering from the M4 or M5 subtype of AML, or a biological sample from a patient suffering from MDS, wherein the number of samples in the population is sufficient to reasonably reflect the distribution of primary RNA transcript levels in a group of patients having the M4 or M5 subtype of AML or MDS that is larger than the population of samples;
the analysis of primary RNA transcript levels in the population comprises testing at least some of the samples for responsiveness to tamibarotene and establishing (i) the lowest primary RNA transcript level of a sample in the population that responds to tamibarotene and (ii) the highest primary RNA transcript level of a sample in the population that does not respond to tamibarotene, thereby defining the lowest RNA transcript responder and the highest RNA transcript non-responder, respectively; and the threshold level is set (i) at a level equal to or up to about 5% above the primary RNA transcript level in the lowest primary RNA transcript responder, (ii) equal to or up to about 5% above the primary RNA transcript level in the highest primary RNA transcript non-responder, or (iii) to a value in between the primary RNA transcript level of the lowest primary RNA transcript responder and the primary RNA transcript level of the highest primary RNA transcript non-responder.

In one embodiment, the present invention features a method of treatment or the use of therapeutically effective amounts of tamibarotene or a pharmaceutically acceptable salt thereof, azacitidine or a pharmaceutically acceptable salt thereof, and venetoclax or a pharmaceutically acceptable salt thereof (e.g., tamibarotene and azacitidine and venetoclax) in treating a patient who has been diagnosed with acute myeloid leukemia (AML) or MDS. This method/use constitutes a fourth specific embodiment of the invention.

In the fourth specific embodiment, the tamibarotene, azacitidine, and venetoclax, or one or more of the salts thereof, are administered to the patient prior to determining whether leukemic cells in a biological sample obtained from the patient expresses a RARA biomarker and/or without consideration of the status of the RARA biomarker. Alternatively, the therapeutically effective amounts of tamibarotene, azacitidine, and ventoclax, or one or more of the salts thereof, are administered to the patient after the patient has been determined to express a RARA biomarker. The RARA biomarker can be elevated expression, relative to a reference, of a RARA primary RNA transcript, a cDNA transcribed from the RARA primary RNA transcript, or a super enhancer associated with the RARA gene. The patient can be newly diagnosed with AML or MDS, and the methods and uses can further comprise administration of a therapeutically effective amount of low-dose cytarabine. Alternatively or in addition, the patient can be considered unfit for standard induction chemotherapy, and the methods and uses can further comprise administration of a therapeutically effective amount of low-dose cytarabine. The RARA biomarker can be assessed by determining whether cancer cells in a biological sample obtained from the patient have (a) a super enhancer associated with a RARA gene, wherein the super enhancer has a strength or an ordinal rank based on its strength or prevalence that is equal to or above a pre-determined threshold level; and/or (b) a level of primary RNA transcript from the RARA gene that is equal to or above a pre-determined threshold level. The RARA biomarker can consist of or comprise:
(a) a primary RNA transcript level from the RARA gene, wherein the transcript level is elevated relative to a threshold level that defines a dividing line between patients who are likely to respond to tamibarotene and patients who are not likely to respond to tamibarotene and is pre-determined by analysis of primary RNA transcript levels in a population of samples comprising a cell line representing AML, a cell line representing MDS, a xenograft representing AML, a xenograft representing MDS, a biological sample from a patient suffering from AML, or a biological sample from a patient suffering from MDS, wherein
the number of samples in the population is sufficient to reasonably reflect the distribution of RARA primary RNA transcript levels in a group of patients having AML or MDS that is larger than the population of samples;
the analysis of RARA primary RNA transcript levels in the population comprises testing at least some of the samples for responsiveness to tamibarotene and establishing (i) the lowest primary RNA transcript level of a sample in the population that responds to tamibarotene and (ii) the highest primary RNA transcript level of a sample in the population that does not respond to tamibarotene, thereby defining the lowest RARA RNA transcript responder and the highest RARA RNA transcript non-responder, respectively; and
the threshold level is set (i) at a level equal to or up to 5% above the RARA RNA transcript level in the lowest RARA RNA transcript responder, (ii) equal to or up to 5% above the RARA RNA transcript level in the highest RARA RNA transcript non-responder, or (iii) to a value in between the RARA RNA transcript level of the lowest RARA RNA transcript responder and the RARA RNA transcript level of the highest RARA RNA transcript non-responder.

In one embodiment, the present invention features a method of treatment or the use of a therapeutically effective amount of tamibarotene or a pharmaceutically acceptable salt thereof in treating a patient who has been diagnosed with chronic myelomonocytic leukemia (CMML), chronic lymphocytic leukemia (CLL (e.g., with 17p deletion)), acute lymphoblastic leukemia (ALL), small lymphocytic lymphoma (SLL), multiple myeloma (MM), non-Hodgkin lymphoma (NHL), or mantle cell lymphoma (MCL). This method/use constitutes a fifth specific embodiment of the invention.

In the fifth specific embodiment, the tamibarotene or the pharmaceutically acceptable salt thereof is administered to the patient prior to determining whether leukemia, lymphoma, or mantle cells in a biological sample obtained from the patient expresses a RARA biomarker and/or without consideration of the status of the RARA biomarker. Alternatively or in addition, the tamibarotene or the pharmaceutically acceptable salt thereof is administered to the patient after determining whether leukemia, lymphoma, or mantle cells in a biological sample obtained from the patient express a RARA biomarker and/or at least one biomarker indicative of a monocytic phenotype. The RARA biomarker can consist of or comprise (i) elevated expression, relative to a reference, of a RARA primary RNA transcript or a cDNA transcribed therefrom, or (ii) a super enhancer associated with the RARA gene and (b) the at least one biomarker indicative of a monocytic phenotype comprises (i) elevated expression, relative to a reference, of a primary RNA transcript from a CD14 gene, a CLEC7A (CD369) gene, a CD86 gene, a CD68 gene, a LYZ gene, an MAFB gene, a CD34 gene, an ITGAM (CD11b) gene, and/or an FCGR1A (CD64) gene, a cDNA transcribed therefrom, or a protein encoded thereby or (ii) a super enhancer associated with the CD14 gene, the CLEC7A (CD369) gene, the CD86 gene, the CD68 gene, the LYZ gene, the MAFB gene, the CD34 gene, the ITGAM (CD11b) gene, the FCGR1A (CD64) gene, a KIT (CD117) gene, the MCL1 gene and/or the BCL2. The tamibarotene or the pharmaceutically acceptable salt thereof can be administered in combination with a therapeutically effective amount of a second therapeutic agent or therapeutically effective amounts of a plurality of additional therapeutic agents. The second therapeutic agent can be a hypomethylating agent (e.g., azacitidine or decitabine). The second therapeutic agent can be a BCL2 inhibitor (e.g., venetoclax). Tamibarotene can be administered in combination with a hypomethylating agent and a BCL2 inhibitor. Tamibarotene can be administered in combination with azacitidine and venetoclax. The patient may be one who has relapsed following treatment with venetoclax or one who has become refractory to treatment with venetoclax. Leukemic cells, lymphoma cells, or myeloma cells within a biological sample obtained from the patient may have demonstrated resistance to venetoclax. The at least one biomarker indicative of a monocytic phenotype can be a gene or protein having a level of expression, relative to a reference, that correlates with resistance to venetoclax, optionally wherein the at least one biomarker indicative of a monocytic phenotype comprises a gene selected from CD14, CLEC7A (CD369), CD86, CD68, LYZ, MAFB, CD34, ITGAM (CD11b), FCGR1A (CD64), or KIT (CD117), MCL1, and BCL2, a cDNA transcribed therefrom, or a protein encoded thereby. The plurality of additional therapeutic agents can consist of or comprise therapeutically effective amounts of azacitidine or decitabine, venetoclax, and obinutuzumab, which can be administered to a patient diagnosed with CLL or SLL. The plurality of additional therapeutic agents can consist of or comprise therapeutically effective amounts of azacitidine or decitabine, venetoclax, and rituximab, which can be administered to a patient diagnosed with CLL or SLL. The RARA biomarker and/or the at least one biomarker indicative of a monocytic phenotype is or has been assessed by determining whether cancer cells in a biological sample obtained from the patient have (a) a super enhancer associated with a RARA gene or a gene indicative of a monocytic phenotype, wherein the super enhancer has a strength or an ordinal rank based on its strength or prevalence that is equal to or above a pre-determined threshold level; and/or (b) a level of primary RNA transcript from the RARA gene or the gene indicative of a monocytic phenotype that is equal to or above a pre-determined threshold level.

The RARA biomarker and the at least one biomarker indicative of a monocytic phenotype can be or can comprise:
(a) a primary RNA transcript level from the RARA gene or a biomarker gene indicative of a monocytic phenotype, wherein the transcript level is elevated relative to a threshold level that defines a dividing line between patients who are likely to respond to tamibarotene and patients who are not likely to respond to tamibarotene and is pre-determined by analysis of primary RNA transcript levels in a population of samples comprising a cell line representing the M4 or M5 subtype of AML, a cell line representing MDS, a xenograft representing the M4 or M5 subtype of AML, a xenograft representing MDS, a biological sample from a patient suffering from the M4 or M5 subtype of AML, or a biological sample from a patient suffering from MDS, wherein
the number of samples in the population is sufficient to reasonably reflect the distribution of primary RNA transcript levels in a group of patients having the M4 or M5 subtype of AML or MDS that is larger than the population of samples;
   the analysis of primary RNA transcript levels in the population comprises testing at least some of the samples for responsiveness to tamibarotene and establishing (i) the lowest primary RNA transcript level of a sample in the population that responds to tamibarotene and (ii) the highest primary RNA transcript level of a sample in the population that does not respond to tamibarotene, thereby defining the lowest RNA transcript responder and the highest RNA transcript non-responder, respectively; and
the threshold level is set (i) at a level equal to or up to about 5% above the primary RNA transcript level in the lowest primary RNA transcript responder, (ii) equal to or up to about 5% above the primary RNA transcript level in the highest primary RNA transcript non-responder, or (iii) to a value in between the primary RNA transcript level of the lowest primary RNA transcript responder and the primary RNA transcript level of the highest primary RNA transcript non-responder.

In the embodiments of the invention, and in particular in the first, second, third, or fourth specific embodiment, the AML is non-APL AML.

Assessing expression of a biomarker: The techniques and analyses described herein and related to assessing expression of a biomarker can apply to any one or more of the specific biomarkers described herein (e.g., a RARA biomarker or a biomarker of the monocytic subtype) unless the context indicates otherwise. One can identify a biomarker described herein (e.g., RARA, CD14, CLEC7A (CD369), CD86, CD68, LYZ, MAFB, CD34, ITGAM (CD11b), FCGR1A (CD64), or KIT (CD117), and biomarkers correlated thereto (e.g., MCL1 and/or BCL2) by identifying and assessing a super enhancer associated with any given biomarker gene. Super enhancers can be identified by various methods known in the art (see, Cell 155:934-947, 2013 and U.S. Pat. No. 9,181,580, the content of which is hereby incorporated by reference herein in its entirety). Identifying a super enhancer can begin by obtaining cellular material comprising DNA from cancer cells within a biological sample obtained from a patient (e.g., a sample of blood or tissue obtained by biopsy). The important metrics for enhancer measurement occur in two dimensions; the length of the DNA over which a genomic marker associated with the super enhancer (e.g., H3K27Ac) can be contiguously detected constitutes the first dimension, and the compiled incidence of genomic marker at each base pair along that length of DNA constitutes the magnitude and serves as the second dimension. The area under the curve (“AUC”) resulting from integration of length and magnitude analysis determines the strength of the enhancer. It is known that the RARA gene is associated with a super enhancer and it is expected that enhancers associated with one or more biomarkers of the monocytic subtype will qualify as super enhancers as well. The strength of the enhancer/super enhancer associated with a biomarker gene, relative to a control, indicates whether a patient is likely to respond to a therapeutic agent (or combination of therapeutic agents) as described herein, with the presence of a super enhancer and its relative strength identifying patients more likely to respond to treatment. As one of ordinary skill in the art would appreciate, if the length of DNA over which the genomic marker is detected is the same for both a biomarker gene (e.g., RARA) and a control gene, then the amount of genomic marker along the length of DNA, relative to that present within the control, will indicate the strength of the enhancer or super enhancer, as the case may be, and may be used alone to determine whether a patient is likely to respond to a therapeutic agent (or combination of therapeutic agents) as described herein.

We have determined through H3K27Ac ChIP-seq methods that there is a super enhancer locus associated with the RARA gene at chr17:38458152-38516681 (genome build hg19). This locus overlaps the RARA gene locus itself and therefore was considered a super enhancer locus associated with that gene because of proximity/overlap. Thus, in some embodiments, determination of the strength of a super enhancer associated with the RARA gene according to the present invention only requires analysis of this specific portion of the genome, as opposed to requiring an analysis of the entire genome.

ChIP-sequencing, also known as ChIP-seq, is used to analyze protein interactions with DNA. ChIP-seq combines chromatin immunoprecipitation (ChIP) with massively parallel DNA sequencing to identify the binding sites of DNA-associated proteins. It can be used to map global binding sites precisely for any protein of interest. Previously, ChIP-on-chip was the most common technique utilized to study these protein-DNA relations. Successful ChIP-seq is dependent on many factors including sonication strength and method, buffer compositions, antibody quality, and cell number; see, e.g., Furey, Nature Reviews Genetics 13, 840-852, 2012; Metzker, Nature Reviews Genetics 11:31-46, 2010; and Park, Nature Reviews Genetics 10:669-680, 2009. Genomic markers other that H3K27Ac that can be used to identify super enhancers using ChIP-seq include P300, CBP, BRD2, BRD3, BRD4, and components of the mediator complex (Loven, et al., Cell, 153(2):320-334, 2013), histone 3 lysine 4 monomethylated (H3K4mel), or other tissue-specific enhancer-tied transcription factors (Smith and Shilatifard, Nat. Struct. Mol. Biol., 21(3):210-219, 2014; Pott and Lieb, Nature Genetics, 47(1):8-12, 2015). H3K27ac or other marker ChIP-seq data super enhancer maps of the entire genome of a cell line or a patient sample already exist in some cases. If so, one would simply determine whether the strength or an ordinal ranking (e.g., an ordinal of strength or prevalence rank) of the enhancer or super enhancer in such maps at the chr17:38458152-38516681 (genome build hg19) locus was equal to or above the pre-determined threshold level for the RARA super enhancer.

In some instances, determination of whether the strength of the enhancer or super enhancer at the chr17:38458152-38516681 locus (i.e., the RARA locus) requires a comparison of the ChIP-seq reads in this region to a region known to comprise a ubiquitous super enhancer or enhancer that is present at similar levels in all cells. One example of such a ubiquitous super enhancer region is the MALAT1 super enhancer locus (chr11:65263724-65266724). By comparing the ChIP-seq reads at the RARA locus (or loci for biomarker genes indicative of the monocytic phenotype) with that at the MALAT1 locus, one can determine whether the strength of a super enhancer at the RARA locus is equal to or above the predetermined threshold level and whether or not the cells therein will respond to a RARA agonist (or whether cancer cells having an enhancer or super enhancer associated with a biomarker of the monocytic phenotype will respond to a therapeutic regimen described herein for patients whose cancers demonstrate a monocytic phenotype (e.g., the M4 or M5 subtype of AML or MDS).

In some embodiments, the pre-determined threshold level is the level at which log₁₀ (AUC of ChIP-seq reads at the biomarker locus (e.g., the RARA locus) (“R”)/AUC of ChIP-seq reads at the MALAT1 super enhancer locus (“M”) is 0.25 or greater. Thus, the threshold level for identifying likely responders to a therapeutic regimen described herein (e.g., responders to tamibarotene, tami/aza, or tami/aza/ven) is log₁₀(R/M) of 0.3 or greater (e.g., 0.35 or greater or 0.4 or greater).

In some embodiments, the pre-determined threshold level is the level at which log₁₀ (AUC of ChIP-seq reads at the biomarker locus (e.g., the RARA locus) (“R”)/AUC of ChIP-seq reads at a MALAT1 super enhancer locus (“M”)) is 1.75 or greater. Thus, the threshold level for identifying likely responders to a therapeutic regimen described herein (e.g., responders to tamibarotene, tami/aza, or tami/aza/ven) is log₁₀(R/M) of 2.0 or greater (e.g., 2.25 or greater or 2.75 or greater).

As indicated, the control enhancer or super enhancer locus can be other than MALAT1. In some embodiments, R, as defined above, is compared to a control enhancer or super enhancer locus other than MALAT1 (the number of ChIP-seq reads at this other control enhancer or super enhancer is referred to as “C”). When another control enhancer or super enhancer locus, C, is utilized, the threshold values expressed as log₁₀ (“V”), referred to above for comparison to M, e.g., log₁₀(R/M) greater than or equal to 0.25, log₁₀(R/M) greater than or equal to 0.3, log₁₀(R/M) greater than or equal to 0.35, or log₁₀(R/M) greater than or equal to 0.4, must be adjusted to an equivalent value to compare to C in order to account for the relative strength of C as compared to M. This “equivalent adjusted threshold value” (“A”) is calculated as follows: A=log₁₀(M/C)+V.

As a non-limiting example, if the calculated strength of the MALAT1 super enhancer (M) is 10-fold greater than the control enhancer or super enhancer used as a comparator (C), and the threshold value (V) is 0.25, then A=log₁₀(10)+0.25=1.25 and the adjusted threshold value is 1.25. For this example, when C is used as the comparator, then log₁₀(R/C) equal or greater than 1.25 is considered the equivalent to a log₁₀(R/M) equal to or greater than 0.25 when M is used as the comparator. It will be readily apparent that an adjusted threshold value can be calculated in a similar manner for any additional comparator based on its relative strength to either MALAT1 or any other comparator for which an adjusted threshold value has already been determined.

The same adjustments above can be made when linear values compared to M are used as threshold levels (e.g., greater than or equal to 1.75-fold, greater than or equal to 2.0-fold, greater than or equal to 2.25-fold, or 2.5-fold). In this case, one obtains the ratio of M to C, and then multiplies the threshold value by that ratio to obtain appropriate threshold values when comparing R to C (i.e., (threshold value)_{C}=(M/C)(threshold value)_{M}).

The specific chromosomal locations of RARA, genes serving as biomarkers of the monocytic phenotype, MALAT1, and other controls may differ for different genome builds and/or for different cell types. However, one of ordinary skill in the art can determine such different locations by locating, in such other genome builds, specific sequences corresponding to the loci in genome build hg 19.

Other methods that can be used to identify a super enhancer include chromatin immunoprecipitation (Delmore et al., Cell, 146(6):904-917, 2011) and chip array (ChIP-chip), and chromatin immunoprecipitation followed by qPCR (ChIP-qPCR) using the same immunoprecipitated genomic markers and oligonucleotide sequences that hybridize to the chr17:38458152-38516681 (genome build hg19) RARA locus. In the case of ChIP-chip, the signal is typically detected by intensity fluorescence resulting from hybridization of a probe and input assay sample as with other array-based technologies. For ChIP-qPCR, a dye that becomes fluorescent only after intercalating the double stranded DNA generated in the PCR reaction is used to measure amplification of the template.

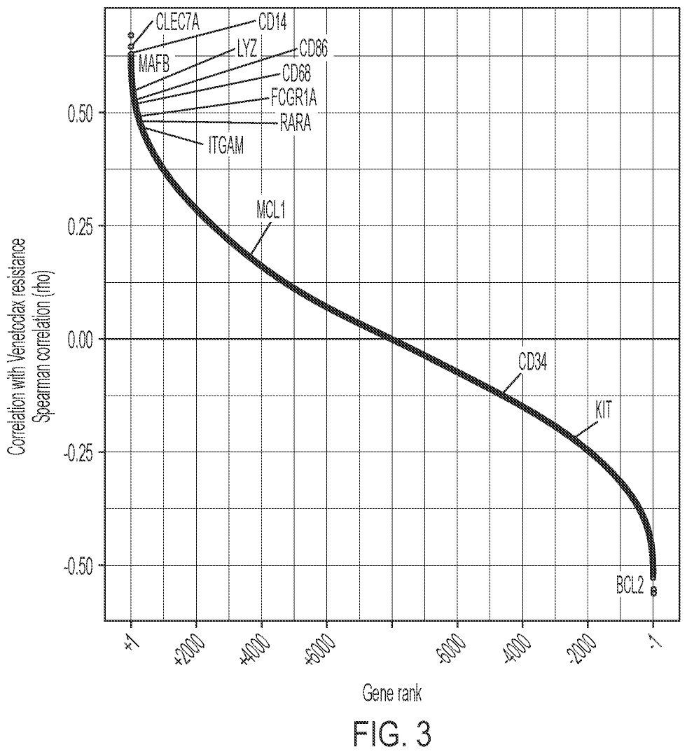

In some embodiments, determining whether a cell expresses a biomarker, as evidenced by the strength of a super enhancer above a requisite threshold level, is achieved by comparing the strength of the enhancer in a test cell (e.g., a cancer cell in a biological sample obtained from a patient) to the strength of a corresponding enhancer in a cell known to not respond to RARA (a “control cell”). Preferably the control cell is the same cell type as the test cell (e.g., a cancer cell in a biological sample obtained from a patient). In some instances, the control cell is such cell in an HCC1143 cell line or any cell listed in FIGS. 3A-3M of U.S. Pat. No. 10,697,025, wherein log₁₀(RARA/MALAT1) is less than 0.25, less than 0.2, less than 0.15, less than 0.1, or less than 0. U.S. Pat. No. 10,697,025 is hereby incorporated by reference herein in its entirety. In some embodiments, a patient is determined to be a likely responder to a RARA agonist (e.g., tamibarotene) if the strength of a biomarker (e.g., a RARA biomarker or biomarker indicative of the monocytic phenotype), as evidenced by the presence of a super enhancer, in cancer cells in a biological sample obtained from the patient, is at least about 1.5-fold greater than the strength of a corresponding enhancer or super enhancer in a control cell (e.g., at least about 2.0, 2.5, 3.0, 4.0 or 5.0 fold greater). In any of these embodiments, the strength of the super enhancer associated with a biomarker in both the test cell(s) (e.g., obtained from the patient) and the control cell(s) can be normalized before comparison. Normalization involves adjusting the determined strength of a super enhancer by comparison to either another enhancer or super enhancer that is native to and present at equivalent levels in both of the cells (e.g., MALAT1) or to a fixed level of exogenous DNA that is purposefully added (“spiked”) into samples of each of the cells prior to determining the strength of the enhancer or super enhancer strength (Orlando et al., Cell Rep. 9(3):1163-70, 2014; Bonhoure et al., Genome Res, 24:1157-68, 2014).

Determining whether a cell (e.g., a cancer cell in a biological sample obtained from a patient) is biomarker-positive by virtue of having a super enhancer strength above a requisite threshold level can be achieved by comparing the strength of the enhancer or super enhancer associated with the biomarker (e.g., RARA or a biomarker gene indicative of a monocytic phenotype) in a test cell (e.g., a cancer cell within a biological sample obtained from the patient) to the strength of the corresponding enhancer or super enhancer in a population of samples, wherein each of the samples in the population of samples is obtained from a different source (i.e., a different subject, a different cell line, a different xenograft). At least some of the samples in the population of samples will have been tested for responsiveness to a specific therapeutic agent (e.g., a RARA agonist such as tamibarotene) in order to establish: (a) the lowest enhancer strength in a sample in the population of samples that responds to that specific therapeutic agent (e.g., tamibarotene) (the “lowest responder”); and, optionally, (b) the highest enhancer strength in a sample in the population of samples that does not respond to that specific therapeutic agent (e.g., tamibarotene; the “highest non-responder”). In these embodiments, a threshold level or “cutoff” of enhancer strength (e.g., RARA SE strength) above which a test cell would be considered responsive to that specific therapeutic agent (e.g., tamibarotene) is set: (i) equal to or up to about 5% above the enhancer strength in the lowest responder in the population; or (ii) equal to or up to about 5% above the enhancer strength in the highest non-responder in the population; or (iii) to a value in between the enhancer strength of the lowest responder and the highest non-responder in the population.

It should be understood that in the above embodiments typically not all of the samples in a population need to be tested for responsiveness to the RARA agonist, but all samples are measured for RARA enhancer strength. In some embodiments, the samples are rank ordered based on RARA enhancer strength. The choice of which of the three methods set forth above to use to establish the cutoff will depend upon the difference in RARA enhancer strength between the lowest responder and the highest non-responder in the population and whether the goal is to minimize the number of false positives or to minimize the chance of missing a potentially responsive sample or subject. When the difference between the lowest responder and highest non-responder is large (e.g., when there are many samples not tested for responsiveness that fall between the lowest responder and the highest non-responder in a rank ordering of RARA enhancer strength), the cutoff is typically set equal to or up to 5% above the RARA enhancer strength in the lowest responder in the population. This cutoff maximizes the number of potential responders. When this difference is small (e.g., when there are few or no samples untested for responsiveness that fall between the lowest responder and the highest non-responder in a rank ordering of RARA enhancer strength), the cutoff is typically set to a value in between the RARA enhancer strength of the lowest responder and the highest non-responder. This cutoff minimizes the number of false positives. When the highest non-responder has a RARA enhancer strength that is greater than the lowest responder, the cutoff is typically set to a value equal to or up to 5% above the RARA enhancer strength in the highest non-responder in the population. This method also minimizes the number of false positives.

Determining whether a cell has a super enhancer (e.g., RARA SE) above a requisite threshold level can be achieved by comparing the ordinal of enhancer strength in a test cell to the ordinal of enhancer strength (for the same enhancer) in a population of cell samples, wherein each of the cell samples is obtained from a different source (i.e., a different subject, a different cell line, a different xenograft). In these embodiments, at least some of the samples in the population will have been tested for responsiveness to a specific therapeutic agent (e.g., a RARA agonist such as tamibarotene) in order to establish: (a) the lowest enhancer strength ordinal of a sample in the population that responds to that specific therapeutic agent (“lowest ordinal responder”); and, optionally, (b) the highest enhancer strength ordinal of a sample in the population that does not respond to that specific therapeutic agent (“highest ordinal non-responder”). In these embodiments, a cutoff of enhancer strength ordinal (e.g., RARA SE strength ordinal) above which a test cell (e.g., a cancer cell from a biological sample obtained from a patient) would be considered responsive to that specific therapeutic agent is set: (i) equal to or up to about 5% above the enhancer strength ordinal in the lowest ordinal responder in the population; or (ii) equal to or up to about 5% above the enhancer strength ordinal in the highest ordinal non-responder in the population; or (iii) to a value in between the enhancer strength ordinal of the lowest ordinal responder and the highest ordinal non-responder in the population. Not all the samples in a population need to be tested for responsiveness to the therapeutic agent (e.g., a RARA agonist), but the enhancer strength and the ordinal of enhancer strength compared to other enhancers in the same sample is measured in all, or essentially all, of the samples. The ordinal is typically obtained by measuring the strength of all, or essentially all, other enhancers in the cell and determining what rank (i.e., the ordinal) in terms of strength the enhancer (e.g., the RARA enhancer) has as compared to the other enhancers (i.e., enhancers associated with genes other than the RARA gene or, as the analysis may dictate, a biomarker gene indicative of a monocytic phenotype). In some embodiments, the samples are rank ordered based on the ordinal of enhancer strength (e.g., RARA SE strength). The choice of which of the three methods set forth above ((i)-(iii)) to use to establish the pre-determined threshold or cutoff will depend upon the difference in the strength or the ordinals of enhancer strength between the lowest ordinal responder and the highest ordinal non-responder in the population and whether the threshold or cutoff is designed to minimize false positives or maximize the number of responders. When this difference is large (e.g., when there are many samples not tested for responsiveness that fall between the lowest ordinal responder and the highest ordinal non-responder in a rank ordering of ordinals of enhancer strength), the cutoff is typically set equal to or up to about 5% above the ordinal of enhancer strength in the lowest ordinal responder in the population. When this difference is small (e.g., when there are few or no samples untested for responsiveness that fall between the lowest ordinal responder and the highest ordinal non-responder in a rank ordering of the ordinal of enhancer strength), the cutoff is typically set to a value in between the ordinal of enhancer strength of the lowest ordinal responder and the highest ordinal non-responder. When the highest ordinal non-responder has an ordinal of enhancer strength that is greater than that of the lowest responder, the cutoff is typically set to a value equal to or up to about 5% above the ordinal of RARA enhancer strength in the highest ordinal non-responder in the population.

Where a test cell (e.g., cancer cells in a biological sample obtained from a patient) is compared to a population, the cutoff value(s) obtained for the population (e.g., RARA enhancer strength or RARA enhancer ordinal) can be converted to a prevalence rank and the threshold or cutoff is expressed as a percent of the population having the threshold or cutoff value or higher (i.e., a prevalence cutoff). Without being bound by theory, the Applicants believe that the prevalence rank of a test sample will be similar regardless of the methodology used to determine enhancer strength. Thus, a prevalence cutoff determined for one parameter (e.g., RARA enhancer strength ordinal) is portable and can be applied to another parameter (e.g., RARA mRNA level) to determine the cutoff value for that other parameter. This allows the determination of a cutoff value for any parameter without having to experimentally determine the correlation between levels of such parameter and responsiveness to the RARA agonist. All that needs to be determined is what level of such other parameter corresponds to the prior determined prevalence cutoff in a population.

Determining Levels of Primary RNA Transcripts: We have shown that levels of mRNA transcripts encoding RARA correlate with sensitivity to RARA agonists (e.g., tamibarotene), and thus RARA RNA transcript levels can be used to identify cells (e.g., cancer cells within a biological sample obtained from a patient) that are likely to respond to RARA agonists (e.g., tamibarotene) and therapeutic regimens including it (e.g., tami/aza and tami/aza/ven). We expect cancer cells having a monocytic phenotype to carry a super enhancer associated with the RARA gene and to express elevated levels of RARA primary RNA transcripts. Thus, determining a monocytic phenotype (by, for example, a technique described herein, including determining whether cells in question express an MES) identifies cells likely to respond to RARA agonists (e.g., tamibarotene) and therapeutic regimens including it (e.g., tami/aza and tami/aza/ven).

Primary RNA transcript levels from the super enhancer locus can be determined using quantitative techniques that compare primary RNA transcript levels (from, e.g., the RARA gene or a biomarker indicative of monocytic phenotype) in a sample with corresponding primary RNA transcript levels in a population of cells (e.g., a cell line) known to be non-responsive to a RARA agonist (e.g., tamibarotene). Such quantitative techniques include RNA array or sequencing-based methods for reading the RNA (e.g., eRNA associated with enhancer read through; see Hah et al., PNAS, 112(3):E297-302, 2015), RNA qPCR, and RNA-Seq. Other methods of quantifying specific primary RNA transcripts in a cell (e.g., cancer cells in a biological sample obtained from a patient) are known in the art and include, but are not limited to, fluorescent hybridization such as utilized in services and products provided by NanoString Technologies, array based technology (Affymetrix), reverse transcriptase qPCR as with SYBR® Green (Life Technologies) or TaqMan® technology (Life Technologies), RNA sequencing (e.g., RNA-seq), RNA hybridization and signal amplification as utilized with RNAscope® (Advanced Cell Diagnostics), or northern blot.

A pre-determined threshold level can be set where the primary RNA transcript level (e.g., transcripts from the RARA gene or a biomarker gene indicative of monocytic phenotype) is at least about 1.5 fold higher (e.g., at least about 2.0, 2.5, 3.0, 4.0, or 5.0-fold higher) than that of a corresponding primary RNA transcript level in a population of cells (e.g., a cell line) that is non-responsive to a RARA agonist (e.g., tamibarotene). The non-responsive population of cells may be referred to as the control population, and cells of the cell line HCC1143 are useful in that regard. Test cells (e.g., cancer cells in a biological sample obtained from a patient) expressing such a level of primary RNA transcripts have reached or exceeded a threshold level that identifies them as likely responders to the RARA agonist.

In determining the threshold level, at least some of the samples in the population of sample will have been tested for responsiveness to a therapeutic agent (e.g., a RARA agonist such as tamibarotene) to establish: (a) the lowest primary RNA transcript (e.g., mRNA) level in a sample in the population that responds to that specific therapeutic agent (the “lowest primary RNA transcript responder”); and, optionally, (b) the highest primary RNA transcript (e.g., mRNA) level in a sample in the population that does not respond to that specific therapeutic agent (the “highest mRNA non-responder”). A threshold level or “cutoff,” against which data from a test cell can be compared, is then set: (i) equal to or up to about 5% above the primary RNA transcript level in the lowest primary RNA transcript responder in the population; or (ii) equal to or up to about 5% above the primary RNA transcript level in the highest primary RNA transcript non-responder in the population; or (iii) a value in between the primary RNA transcript level of the lowest primary RNA transcript responder and the highest primary RNA transcript non-responder in the population. The RARA gene and any biomarker gene indicative of a monocytic phenotype can be assessed in this way to set a pre-determined threshold level against which data from test cells can be compared. Not all the samples in a population need to be tested for responsiveness to the therapeutic agent (e.g., a RARA agonist), but the primary RNA transcript level of the gene in question (e.g., RARA or a biomarker gene indicative of the monocytic phenotype) is measured in all, or essentially all, of the samples. If desired, the samples can be rank ordered based on the level of the primary RNA transcript assessed or the levels of primary RNA transcripts in a sample can be rank ordered.

The choice of which of the three methods set forth above ((i)-(iii)) to use to establish the pre-determined threshold or cutoff will depend upon the difference in primary RNA transcript levels between the lowest primary RNA transcript responder and the highest primary RNA transcript non-responder in the population and whether the threshold or cutoff is designed to minimize false positives or maximize the potential number of responders. When this difference is large (e.g., when there are many samples not tested for responsiveness that fall between the lowest primary RNA transcript responder and the highest primary RNA transcript non-responder in a rank ordering of RARA mRNA levels), the cutoff is typically set equal to or up to about 5% above the primary RNA transcript level in the lowest primary RNA transcript responder in the population. When this difference is small (e.g., when there are few or no samples untested for responsiveness that fall between the lowest primary RNA transcript responder and the highest primary RNA transcript non-responder in a rank ordering of primary RNA transcript levels), the threshold or cutoff is typically set to a value in between the levels of the lowest primary RNA transcript responder and the highest primary RNA transcript non-responder. When the highest primary RNA transcript non-responder has primary RNA transcript levels that are greater than the lowest primary RNA transcript responder, the cutoff is typically set to a value equal to or up to about 5% above the RARA mRNA levels in the highest mRNA non-responder in the population.

In embodiments where the population of samples is rank ordered based on primary RNA transcript level, the primary RNA transcript level in each sample can be measured and compared to the primary RNA transcript levels of all other primary RNA transcript in the cell to obtain an ordinal ranking of the primary RNA transcript level. A threshold level or cutoff based on an ordinal ranking of the primary RNA transcript level is then determined based on samples in the population tested for responsiveness to a RARA agonist (e.g., tamibarotene) in the same manner as described previously for determining a threshold based on an ordinal ranking of the strength of a super enhancer. The determined primary RNA transcript ordinal cutoff is then used either either directly or, indirectly, to determine a prevalence cutoff, either of which can be used to stratify additional samples for potential responsiveness to the therapeutic agent (e.g., a RARA agonist such as tamibarotene).

In some embodiments, the cutoff for primary RNA transcript levels is determined using the prevalence cutoff established based on enhancer strength or an ordinal ranking of enhancer strength, as described above. For example, a population can be measured for mRNA levels and the prior determined prevalence cutoff can be applied to that population to determine an mRNA cutoff level. A rank-order standard curve of RARA mRNA levels in a population can then be created, and the pre-determined prevalence cutoff is applied to that standard curve to determine the RARA mRNA cutoff level.

Where data from a test cell (e.g., a cancer cell in a biological sample obtained from a patient) is compared to data from a population of samples, the value determined to be a pre-determined threshold or cutoff level (e.g., the value representing the level of primary RNA transcript) in the population of samples can be converted to a prevalence rank indicating the percent of the population of samples that has the cutoff value or higher, i.e., a prevalence cutoff. Without limiting the invention, the Applicant believes the prevalence cutoff in a population will be similar regardless of the methodology used to determine the strength of the super enhancer or the level of expression of a primary RNA transcript from the biomarker gene.

In some embodiments, a patient is identified as a likely responder to a therapeutic agent (e.g., a RARA agonist responder) if the level of primary RNA transcripts from a RARA gene or a biomarker gene indicative of a monocytic phenotype corresponds to a prevalence rank in a population of 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 43%, 42%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, or 20% as determined by comparable primary RNA transcript levels from the RARA gene or a biomarker gene indicative of a monocytic phenotype in the population. The cutoff value can be established based on the prevalence cutoff established for RARA enhancer strength. Alternatively, the cutoff value is established based on the prevalence cutoff established for RARA enhancer strength ordinal. In another embodiment, the cutoff value is established based on RARA primary RNA levels. In more specific embodiments, a cutoff value for breast cancer patients is established based on the prevalence cutoff determined for RARA enhancer strength ordinal, and that prevalence cutoff value is used as to determine the cutoff value for RARA mRNA levels In even more specific aspects of these embodiments, the cutoff value for breast cancer patients is the value determined using a prevalence value of between 50% and 60%, e.g., 50-55%, 55-60%, 50-56%, 50-57%, 51-55%, 51-56%, 51-57%, 52-55%, 52-56%, 52-57%, 53-55%, 54-56%, 53-56%, or 54-55%. In still more specific embodiments, the cutoff value is set using a prevalence value of 55% or of 56%. A cutoff value for AML, patients can be established based on the prevalence value determined for RARA enhancer strength ordinal, and that prevalence value is used to determine the cutoff value for RARA mRNA levels In even more specific aspects of these embodiments, the cutoff value for AML patients is determined using a prevalence cutoff of between 25-45%, e.g., between 25-30%, 25-35%, 25-40%, 30-35%, 30-40%, 35-45%, 35-40%, 31-35%, 32-35%, 33-35%, 34-35%, 31-36%, 32-36%, 33-36%, 34-36%, or 35-36%. In other more specific embodiments, the cutoff value for AML patients is determined using a prevalence value of about 25%, 30%, about 33%, or about 36%.

For a more granular analysis, any population or population of samples analyzed as described herein can be divided into three groups, thereby defining likely responders, partial responders and non-responders. In this event, two pre-determined threshold levels (e.g., two cutoff values or prevalence cutoffs) are set. The partial responder group may include responders and non-responders, as well as those population members whose response to a RARA agonist was not as high as the responder group. In these embodiments, two cutoff values or prevalence cutoffs are determined. This type of stratification may be particularly useful when in a population the highest RARA mRNA non-responder has a RARA mRNA levels that is greater than the lowest RARA mRNA responder. In this scenario the cutoff level or prevalence cutoff between responders and partial responders is set equal to or up to 5% above the RARA mRNA level of the highest RARA mRNA non-responder; and the cutoff level or prevalence cutoff between partial responders and non-responders is set equal to or up to 5% below the RARA mRNA level of the lowest RARA mRNA responder. The determination of whether partial responders should be administered the RARA agonist will depend upon the judgment of the treating physician and/or approval by a regulatory agency.

The level of primary RNA transcript (e.g., mature mRNA) in both the test cell and the control cell or all members of the population are normalized before comparison. Normalization involves adjusting the determined level of a primary RNA transcript by comparison to either another RNA transcript that is native to and present at equivalent levels in both of the cells (e.g., GADPH mRNA, 18S RNA), or to a fixed level of exogenous RNA that is “spiked” into samples of each of the cells prior determining the strength of the super enhancer (Loven et al., Cell, 151(3):476-82, 2012; Kanno et al., BMC Genomics 7:64, 2006; Van de Peppel et al., EMBO Rep 4:387-93, 2003).

In the methods of the invention, where therapeutic amounts of one or more of the therapeutic agents described herein (e.g., tamibarotene; tami/aza; or tami/aza/ven) are administered to a patient, the therapeutic agent(s) can be administered after determining that cancer cells in a biological sample obtained from the patient express at least one biomarker indicative of a monocytic phenotype and/or a RARA biomarker. For example, tamibarotene can be administered after determining cancer cells of the M4 or M5 subtype of AML express a biomarker indicative of a monocytic phenotype or after determining CMML cells express a RARA biomarker. The RARA biomarker can be a super enhancer associated with a RARA gene, wherein the super enhancer has a strength or an ordinal rank based on its strength or prevalence that is equal to above a pre-determined threshold level, or a level of primary RNA transcripts from the RARA gene that is equal to or above a pre-determined threshold level.

A therapeutic method, as described herein (e.g., treatment of the M4 or M5 subtype of AML with tamibarotene or treatment of any subtype of AML or MDS with a combination of tamibarotene, azacitidine, and venetoclax) can be carried out when the affected (i.e., cancerous) cells in a biological sample obtained from the patient (a) have a super enhancer associated with a RARA gene that is at least about 1.5-fold (e.g., at least about 1.75-fold) stronger than (i) an enhancer or super enhancer associated with a RARA gene in a human cell, human tissue, or human cell line known to be non-responsive to a RARA agonist or (ii) a portion of a super enhancer associated with MALAT1 as measured by ChIP-seq, wherein the portion is located at chr11:65263724-65266724 in genome build hg19, or is at least an equivalent amount stronger than another reference enhancer or super enhancer locus or (b) a RARA primary RNA transcript level that is at least about 1.5-fold higher than the RARA primary RNA transcript level in a human cell, human tissue, or human cell line known to be non-responsive to a RARA agonist.

A therapeutic method, as described herein (e.g., treatment of the M4 or M5 subtype of AML with tamibarotene or treatment of these and any other subtype of AML or MDS with a combination of tamibarotene, azacitidine, and venetoclax) can be carried out when the affected (i.e., cancerous) cells in a biological sample obtained from the patient have (a) a super enhancer associated with a biomarker gene (e.g., a RARA gene) that (i) has a strength greater than or equal to a pre-determined threshold level set by analyzing the strength of a super enhancer driving the expression of the biomarker gene (e.g., the RARA gene) in a population of samples; (ii) has a strength corresponding to an ordinal that is greater than or equal to a pre-determined threshold level set by analyzing and ranking the strength of a super enhancer driving the expression of the biomarker gene (e.g., the RARA gene) in a population of samples; and/or (iii) has a strength corresponding to a prevalence level that is greater than or equal to a pre-determined threshold level set by analyzing and ranking the prevalence of the strength of a super enhancer driving the expression of the biomarker gene (e.g., the RARA gene) in a population of samples; and/or (b) a level of primary RNA transcripts from the biomarker gene (e.g., the RARA gene) that is greater than or equal to a pre-determined threshold level set by analyzing the level of primary RNA transcripts from the biomarker gene (e.g., the RARA gene) in a population of samples. As with the strength of the super enhancer, the level of primary RNA transcripts may be assigned an ordinal and assessed relative to a pre-determined threshold level in an ordinal ranking of the levels of transcripts or the prevalence of the levels of transcripts in a population of samples. The population of samples and the means by which the pre-determined threshold levels can be set are described further herein.

Any of the pre-determined threshold levels can be determined by, first, rank ordering the strength of a super enhancer driving the expression of the biomarker gene (e.g., the RARA gene) in a population of samples or the prevalence of its strength in a population of samples, wherein at least one of the samples has been determined to be sensitive to the effects of the therapeutic agent (e.g., a RARA agonist (e.g., tamibarotene)). Alternatively, or in addition, the pre-determined threshold levels can be determined by, first, rank ordering the levels of primary RNA transcripts from the biomarker gene (e.g., the RARA gene) in a population of samples wherein at least one of the samples has been determined to be sensitive to the effects of the therapeutic agent (e.g., a RARA agonist (e.g., tamibarotene)). The levels can be ranked according to the amount of transcript expressed or the prevalence of the levels of transcripts.

In each embodiment of the present methods, the biomarker in the sample obtained from the patient is or can be the same as the biomarker assessed in the population of samples, and the type of cancer suffered by the patient to be treated is or can be the same as the type of cancer represented by the samples within the population of samples.

In each embodiment of the present methods, determining the status of a biomarker (e.g., RARA or a biomarker of the monocytic phenotype) can be achieved by receiving information related to the strength, ordinal rank or prevalence rank of a super enhancer driving the expression of the biomarker gene in cancer cells within a biological sample obtained from the subject and/or receiving information related to the level of expression of a primary RNA transcript expressed from the biomarker gene in cancer cells within a biological sample obtained from the subject. That information, whether received from a source or actively and independently generated, is compared to a pre-determined threshold, and one or more of the therapeutic agents described herein are administered to the patient when the information indicates that cancer cells within a biological sample obtained from the patient include (a) a super enhancer that drives the expression of a biomarker gene and has a strength, ordinal rank, or prevalence rank that is greater than or equal to a pre-determined threshold level or (b) a level of primary RNA transcripts expressed from the biomarker gene that is greater than or equal to a pre-determined threshold level. Information related to the level of expression of primary RNA transcripts can be provided or secured by (a) obtaining primary RNA transcripts, essentially in total, from a biological sample obtained from the subject; (b) appending to the primary RNA transcripts additional nucleotides that are not naturally appended to the transcripts and that enable the transcripts to bind to a solid support; (c) sequencing the primary RNA transcripts; and (d) determining the level of the primary RNA transcripts. Alternatively, the information can be provided or secured by (a) obtaining primary RNA transcripts, essentially in total, from a biological sample obtained from the subject; (b) creating a cDNA library from the total primary RNA transcripts; and (c) combining the cDNA library with a pair of primers that selectively bind cDNA corresponding to the primary RNA transcript of interest (e.g., that encoded by the RARA gene or a biomarker of the monocytic phenotype); a DNA polymerase; and a component (e.g., a dye or radiolabelled nucleotides) for detecting DNA molecules produced by synthesis allowed by the primer pair and the DNA polymerase.

Determining a cancer type or subtype: The cancer type or subtype (e.g., AML or MDS; ALL, CMML, CLL, SLL, MM, NHL, and MCL) can be determined by one of ordinary skill in the art (i.e., diagnosed) using techniques known in the art. For example, the cancer type or subtype can be determined by assessing blood test results (e.g., for elevated white blood cell counts), genetic tests (for mutations, duplications, deletions, chromosomal rearrangements, and the like), biopsy results (e.g., microscopic examination of blood, bone marrow or other tissue samples), and x-ray and other imaging tests (e.g., echocardiograms, mammograms, and the like). The cancer sub-type (e.g., the M4 or M5 subtype of AML) can be determined by one of ordinary skill in the art (i.e., diagnosed) by assessing a gene or protein expression profile in a biological sample comprising cancer cells from the patient, as described herein, and/or by assessing other defining characteristics, signs, or symptoms of the patient's disease and thereby determining the cancer type in question. Patients who are diagnosed with a cancer type described here are amenable to treatment as described in the embodiments of the invention, and in particular in the first, second, third, fourth and fifth specific embodiments.

Patients diagnosed with the M4 or M5 subtype of AML: A physician may use any credible test or technique (e.g., a standardized and/or FDA-approved test) to diagnose the M4 and M5 subtypes of AML. Generally, factors known to affect staging of blood cancers and prognosis include white blood cell counts, platelet counts, the patient's age and any history of prior blood disorders, mutations or other chromosomal abnormalities, bone damage, and enlargement of the liver or spleen. The French-American-British (FAB) and World Health Organization (WHO) classification systems can be used to determine the subtype of AML in any given patient. The FAB system was devised by experts based on the type of cell giving rise to the leukemia and the maturity of the cells. This system is dependent on microscopic examination of stained cells and includes the following subtypes: M0, also known as undifferentiated acute myeloblastic leukemia; M1, also known as acute myeloblastic leukemia with minimal maturation; M2, also known as acute myeloblastic leukemia with maturation; M3, also known as acute promyelocytic leukemia (APL); M4, also known as acute myelomonocytic leukemia; M4 eos, also known as acute myelomonocytic leukemia with eosinophilia; M5, also known as acute monocytic leukemia; M6, also known as acute erythroid leukemia; and M7, also known as acute megakaryoblastic leukemia. Subtypes M0 through M5 arise in immature forms of white blood cells. The M6 subtype arises from red blood cells, and the M7 subtype arise from precursors to platelet cells. The WHO system includes factors now known to affect prognosis and includes a group defined as “AML not otherwise specified.” This group is similar to the FAB classification just described. Where complete remission (or a complete response) is achieved after treatment, the bone marrow contains fewer than 5% blast cells, blood cell counts are within normal limits, and there are no signs or symptoms from the leukemia. Patients who are diagnosed with a cancer type described here are amenable to treatment as described in the embodiments of the invention, and in particular in the first, second, and fourth specific embodiments.

Patients diagnosed with MDS: A physician may use any credible test or technique (e.g., a standardized and/or FDA-approved test) to diagnose MDS. The FAB system describes MDS cells in terms of five subtypes based on the percentage of blasts in the bone marrow and the peripheral blood as shown in the table below.

| | | % blast in bone |
|---|---|---|
| FAB subtype of MDS | % blasts in blood | marrow |
| Refractory Anemia (RA) | <1 | <5 |
| Refractory Anemia with ringed | <1 | <5 |
| sideroblasts (RARS) | | |
| Refractory Anemia with | <5 | 5-20 |
| excess blasts (RAEB) | | |
| Refractory Anemia with excess | <5 | 21-30 |
| blasts in transformation RAEB-T) | | |
| Chronic myelomonocytic | <5 | 5-20 |
| leukemia (CMMoL) | | |

The WHO classification system expands on the FAB system, by dividing MDS into eight subtypes based on tests of the blood and bone marrow. These eight subtypes include: (1) MDS with single lineage dysplasia (MDS-SLD); (2) MDS with multilineage dysplasia (MDS-MLD); (3) MDS with ring sideroblasts (MDS-RS; including MDS-RS and multilineage dysplasia (MDS-RS-MLD) and MDS-RS and single lineage dysplasia (MDS-RS-SLD)); (4) MDS with Excess Blast (including MDS with Excess Blast-1 and MDS with Excess Blast-2); (5) MDS with isolated del(5q); (6) MDS-unclassifiable (MDS-U); (7) Refractory cytopenia of childhood (RCC, provisional entity); and (8) Myeloid neoplasms with germline predisposition. Patients who are diagnosed with a cancer type described here are amenable to treatment as described in the embodiments of the invention, and in particular in the first, third, and fourth specific embodiments.

Therapeutic agents: In one embodiment, the present methods comprise administering a RARA agonist (e.g., tamibarotene) alone, or in combination, as a “first” agent, with one or more of the “second” therapeutic agents described herein (e.g., a RARA agonist such as tamibarotene can be administered in combination with an HMA, such as azacitidine or decitabine, optionally with the further inclusion of venetoclax, optionally with the further inclusion of LDAC, obinutuzumab (e.g., for patients with CLL or SLL), rituximab (e.g., for patients with CLL or SLL, with or without 17p deletion), or an endocrine therapy. For example, a RARA agonist alone (e.g., tamibarotene), a RARA agonist (e.g., tamibarotene) in combination with an HMA (e.g., azacitidine or decitabine), or a RARA agonist (e.g., tamibarotene) in combination with an HMA (e.g., azacidine or decitabine) and venetoclax, optionally further including LDAC, can be administered to a patient having AML (e.g., a newly diagnosed AML patient of the M4 or M5 subtype (identified by any method known in the art, including by virtue of an MES as described herein) who are at least 60 years old (e.g., 60, 65, 70, or 75 years old or older) or who have comorbidities that preclude use of intensive induction chemotherapy). In another embodiment, the methods comprise administering a RARA agonist alone or a combination of therapeutic agents as just described to a patient who has relapsed following treatment with venetoclax, has become refractory to treatment with venetoclax, or whose cancer cells demonstrate resistance to venetoclax (e.g., by an ex vivo assay). In another embodiment, the methods comprise administering a RARA agonist (e.g., tamibarotene) alone or a combination of a RARA agonist (e.g., tamibarotene) and venetoclax, optionally also including an HMA (e.g., azacitidine or decitabine) and/or obinutuzumab to a patient (e.g., an ND patient) who has CLL or SLL. In another embodiment, the methods comprise administering a RARA agonist (e.g., tamibarotene) alone or a combination of a RARA agonist (e.g., tamibarotene) and venetoclax, optionally also including an HMA (e.g., azacitidine or decitabine) and/or rituximad to a patient (e.g., an ND patient) who has CLL or SLL. In another embodiment, the methods comprise administering a RARA agonist (e.g., tamibarotene) alone or a combination of a RARA agonist (e.g., tamibarotene) and venetoclax, optionally also including an HMA (e.g., azacitidine or decitabine) and/or an endocrine therapy (e.g., tamoxifen) to a patient (e.g., an ND patient) who has a breast cancer (e.g., an ER-positive, BCL2-positive breast cancer, optionally with metastases). Patients who are treated with a therapeutic agent described here are amenable to treatment as further described in the embodiments of the invention, and in particular in the first, second, third, fourth, and fifth specific embodiments.

In any embodiment of the methods described herein, including the methods of the first and fourth specific embodiments, where the patient is suffering from AML, the AML can be a non-acute promyelocytic leukemia acute myelogenous leukemia (i.e., non-APL AML). APL is also known as the M3 subtype of AML under the FAB categorization. Thus, any of the methods described herein can exclude the treatment of APL or exclude AML patients having the M3 subtype of AML. In general, each therapeutic agent (e.g., tamibarotene, azacitidine, decitabine, and venetoclax) for use as described herein is formulated, dosed, and administered in a therapeutically effective amount using pharmaceutical compositions and dosing regimens that are consistent with good medical practice and appropriate for the relevant agent(s) and patient(s). A RARA agonist, venetoclax, and other therapeutic agents described herein may be administered orally, in a formulation and/or amount currently known in the art.

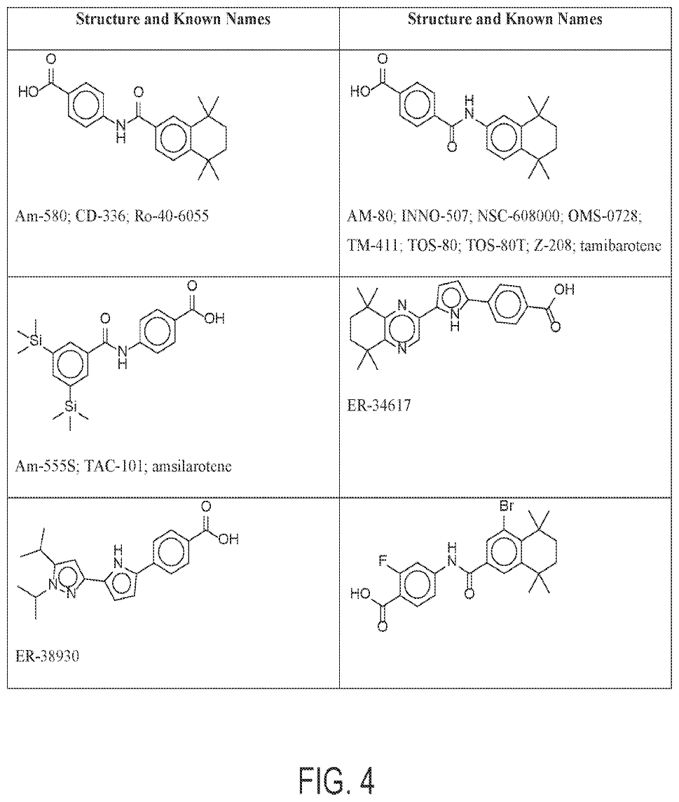

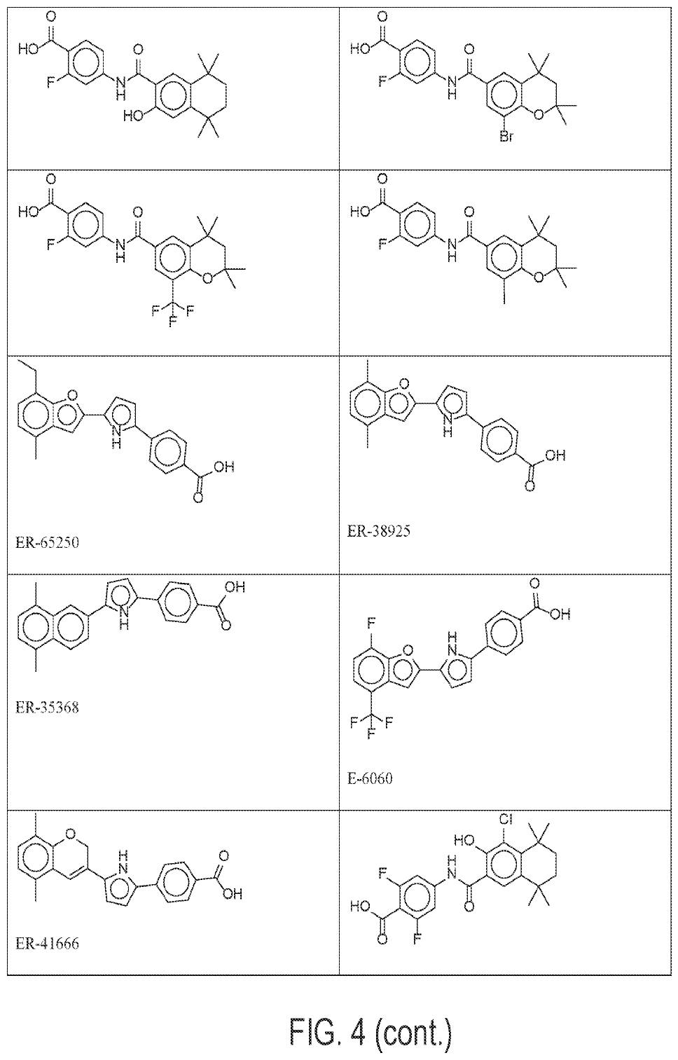

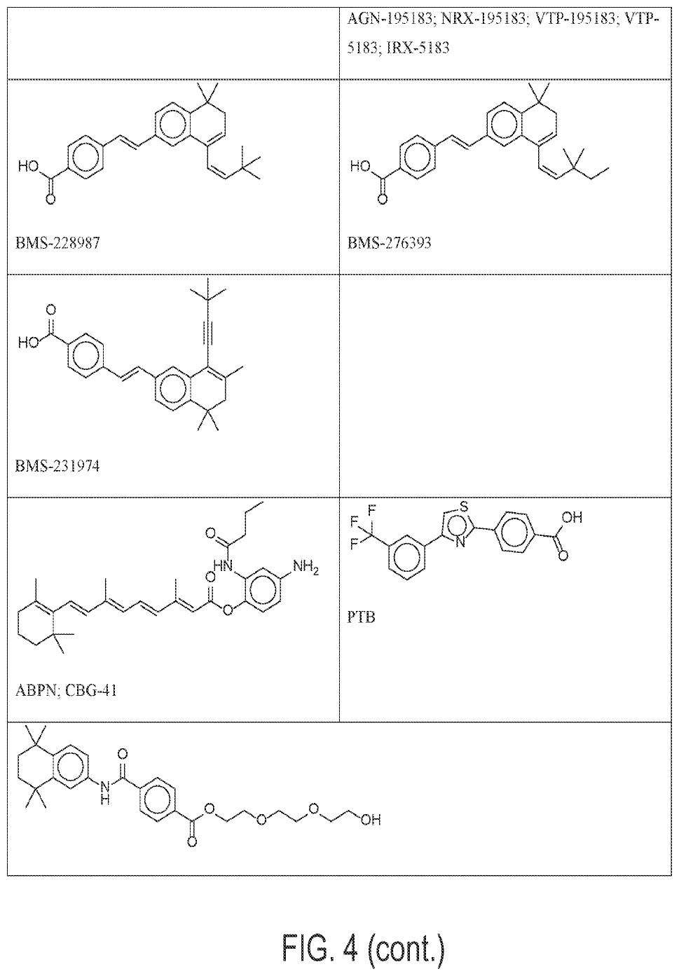

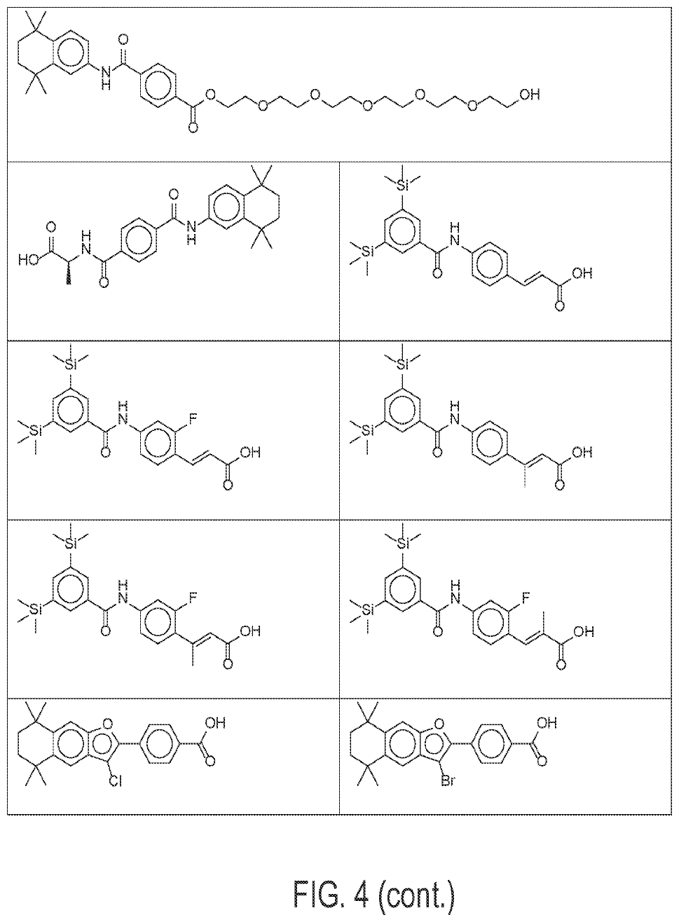

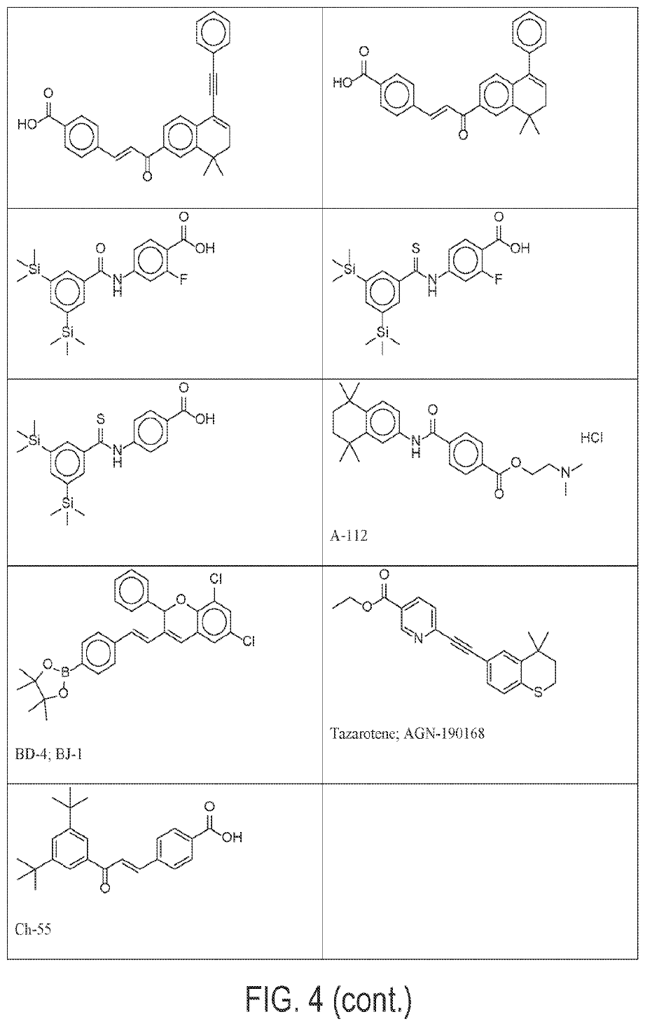

RARA Agonists: In some embodiments, the RARA agonist is selected from a compound disclosed in or any compound falling within the genera set forth in any one of the following United States patents: U.S. Pat. Nos. 4,703,110, 5,081,271, 5,089,509, 5,455,265, 5,759,785, 5,856,490, 5,965,606, 6,063,797, 6,071,924, 6,075,032, 6,187,950, 6,355,669, 6,358,995, and 6,387,950, each of which is hereby incorporated by reference herein in its entirety. Useful RARA agonists are also shown in the Table of FIG. 4. In any of the present methods (i.e., regardless which precise disease or cancer type is being treated (e.g., an M4 or M5 sub-type of AML or MDS), regardless of the patient's prior history (e.g., regardless of whether the patient is newly diagnosed, fit, unfit (e.g., by virtue of being elderly), or experiencing a relapse of their cancer), regardless of the precise manner by which a patient has been diagnosed (e.g., exactly which biomarkers are determined or selected for consideration, if any), and/or regardless of the precise therapeutic agent or combination of therapeutic agents administered), the RARA agonist can be one that selectively binds the alpha form of the receptor (i.e., the RARA agonist will bind RARA preferentially relative to RAR-beta and RAR-gamma). A RARA-selective agonist is at least 10×, 100×, 1000×, or 10000×more specific for RARA than either of RAR-beta or RAR-gamma. Natural ligands such as all-trans retinoic acid (ATRA) and 9-cis retinoic acid may be useful in the present methods, but they are non-selective with regard to the type of RAR they bind and, therefore, may have pleiotropic effects throughout the body, and toxicity may be harder to manage in dosing regimens.

In some embodiments, including the methods of the first embodiment, a RARA agonist (e.g., tamibarotene) is administered (or used) according to a regimen described here. The regimen can include at least one dose (or includes or consists of exactly one or two doses) of about: 1 mg/m² or 1 mg; 2 mg/m² or 2 mg; 3 mg/m² or 3 mg; 4 mg/m² or 4 mg; 5 mg/m² or 5 mg; 6 mg/m² or 6 mg; 7 mg/m² or 7 mg; 8 mg/m² or 8 mg; 9 mg/m² or 9 mg; 10 mg/m² or 10 mg; 11 mg/m² or 11 mg; 12 mg/m² or 12 mg; 13 mg/m² or 13 mg; 14 mg/m² or 14 mg; 15 mg/m² or 15 mg; or 16 mg/m² or 16 mg; or a dose between any two of these values administered once or twice per day. A RARA agonist (e.g., tamibarotene) can be administered at a dose of about 6 mg/m² or 6 mg (e.g., about 6 mg/m²/day or 6 mg/day), about 4 mg/m² or 4 mg (e.g., about 4 mg/m² or 4 mg once or twice per day), about 2 mg/m² or 2 mg (e.g., about 2 mg/m² or 2 mg once or twice per day) or about 1 mg/m² or 1 mg (e.g., about 1 mg/m² or 1 mg once or twice per day). Thus, a dosing regimen may include a plurality of doses, and where a RARA agonist (e.g., tamibarotene) is administered, the methods may include administration of a dose described herein once a day or twice a day. For example, a patient as described herein may be treated such that a total dose of about 6 mg/m² or about 6 mg in total (regardless of body surface area) of tamibarotene is administered daily, optionally divided equally into two doses per day. A patient (e.g., an adult human) having a hematopoietic cancer described herein (i.e., a subtype of AML or non-APL AML, MDS, ALL, CMML, CLL, SLL, MM, NHL, or MCL) can be treated with tamibarotene, administered orally at a dose of 6 mg twice per day on each of days 8-28 of a 28-day treatment cycle. Where doses are administered twice per day, they can be administered, for example, about 12 hours apart, such as around 8 am and 8 pm. The first daily dose and the second daily dose can include equal or unequal amounts of tamibarotene or a pharmaceutically acceptable salt thereof. For example, each dose can contain about 6 mg of tamibarotene. With respect to tableting, the about 6 mg can be contained in a single tablet or multiple tablets (e.g., in two tablets, each containing about 3 mg or in three tablets, each containing about 2 mg of the therapeutic agent). Tamibarotene or the pharmaceutically acceptable salt thereof can be administered orally, and the fixed dose (e.g., the 12 mg total dose, orally, in divided doses) can be administered in any aspect or embodiment of the invention regardless of the patient's weight or body surface area. Patients who are treated with a RARA agonist as described here are amenable to treatment as described in other embodiments of the invention, and in particular in the first, second, third, fourth, and fifth specific embodiments.

Where a RARA agonist (e.g., tamibarotene) is administered in combination with one or more second therapeutic agents, the RARA agonist and/or the one or more second therapeutic agents can be administered according to a dosing regimen for which they are approved for individual use. In any embodiment in which a RARA agonist (e.g., tamibarotene) is administered in combination with an HMA (e.g., azacitidine) and, optionally, a third agent (e.g., venetoclax), the HMA (e.g., azacitidine) can be administered at a dose of about 75 mg/m² (e.g., by a parenteral route of administration such as by an intravenous infusion or subcutaneous injection), once or twice per day, and the RARA agonist (e.g., tamibarotene) can be administered at a dose of about 3-6 mg/m² (e.g., by oral administration), once or twice per day (e.g., about 6 mg/m²/day or 6 mg/day). In the treatment regimen as a whole, the HMA can be administered (e.g., to a patient having AML (e.g., a relapsed or refractory AML)), e.g., in a dose and by a route just described, on days 1-7 of the treatment regimen, and the RARA agonist (e.g., tamibarotene) can be administered, e.g., in a dose and by a route just described, on days 8-28 of the treatment regimen, after which treatment would cease or the patient would have a reprieve from treatment for a period of days or weeks.

HMAs: The HMAs azacitidine and decitabine are valuable options for (but are not limited to treatment of) AML patients who are not eligible for intensive chemotherapy. Azacitidine is FDA-approved in the United States for the treatment of all subtypes of AML, and it is EMA-approved in Europe for treating adult cancer patients who are ineligible for hematopoietic stem cell transplantation. The approved starting dose of 75 mg/m²/day, administered intravenously or subcutaneously on days 1-7 of each 28-day cycle of therapy, can be employed in the methods described herein, including the methods of the first embodiment. Decitabine (5-aza-2′-deoxycytidine) is FDA-approved in the United States for the treatment of patients with MDS. There are two approved dosage regimens, either of which (or others) can be employed in the methods described herein, including the methods of the first embodiment. The first is a three-day regimen, recommended for a minimum of four treatment cycles, in which decitabine at about 15 mg/m² is infused intravenously over three hours every eight hours for three consecutive days. The second is a five-day regimen in which decitabine at about 20 mg/m² is infused intravenously over 1 hour once daily for five consecutive days, every four weeks. Patients who are treated with an HMA as described here are amenable to treatment as described in other embodiments of the invention, and in particular in the first, second, third, fourth, and fifth specific embodiments.

Bcl-2 Inhibitors: Bcl-2 inhibitors, including those approved for use or in clinical-stage development, can be employed in the methods described herein, including the methods of the first embodiment. More specifically, venetoclax is available and can be administered in tablet form, each dosage unit containing either 10, 50, or 100 mg of the therapeutic agent. Where venetoclax is administered in combination with a RARA agonist (e.g., tamibarotene) and an HMA (e.g., azacitidine) to treat a hematological cancer (e.g., CLL or SLL), the venetoclax can be dosed according to a weekly ramp-up schedule over a period of weeks (e.g., five weeks) to the recommended daily dose of 400 mg. For example, a patient with a hematological cancer (e.g., CLL or SLL) may receive a combination therapy as described herein that includes venetoclax at 20 mg, PO, QD in week 1; venetoclax at 50 mg, PO, QD in week 2; venetoclax at 100 mg PO, QD in week 3; venetoclax at 200 mg PO, QD in week 4; and venetoclax at 400 mg PO, QD in week five and beyond. Modified versions of this treatment regimen are known in the art for subsequent cycles of treatment (e.g., cycle 2, cycles 3-6, and cycles 7-12). Dosing of venetoclax can be as described in any one or more of U.S. Pat. Nos. 8,546,399; 8,722,657; 9,174,982; 9,539,251; 10,730,873; and 10,993,942, which are hereby incorporated by reference herein in their entireties. Alternatively, the second-generation Bcl-2 inhibitor S65487 can be administered (e.g., intravenously administered) and may be especially well suited for administration to patients diagnosed with AML, NHL, MM, and CLL. Alternatively, the selective Bcl-2 inhibitor BGB-11417 can be administered (e.g., orally administered once daily) and may be especially well suited for administration to patients diagnosed with relapsed/refractory NHL, CLL, or SLL. Alternatively, the BCL-XL/BCL-2 inhibitor navitoclax (ABT-263) can be administered (e.g., by way of an oral tablet or solution dose of 150 mg lead-in dose for 7-14 days followed by a 325 mg continuous once daily dose) and may be especially well suited for patients diagnosed with NHL or CLL. Alternatively, the Bcl-2 inhibitor pelcitoclax (APG-1252) can be administered (e.g., twice per week (BIW) or once per week (QW) at a dose ranging from 10 to 400 mg in a 28-day cycle) and may be especially well suited for patients diagnosed with NHL. Alternatively, the Bcl-2 inhibitor lisaftoclax (APG-2575) can be administered (e.g., orally at doses ranging from to 1,200 mg) and may be especially well suited for patients diagnosed with R/R CLL. Patients who are treated with a Bcl-2 inhibitor as described here are amenable to treatment as described in other embodiments of the invention, and in particular in the first, second, third, fourth, and fifth specific embodiments.

For administration (either oral or parenteral administration), a therapeutic agent described herein can be readily formulated by combining the agent with one or more pharmaceutically acceptable carriers, which are well known in the art. Indeed, formulations of RARA agonists (e.g., tamibarotene), HMAs (e.g., azacitidine and decitabine), and BCL2 inhibitors (e.g., venetoclax) are known in the art and can be employed in the methods (or used) as described herein, including the methods of the first embodiment. For example, venetoclax is available in tablet form, each dosage unit containing either 10, 50, or 100 mg of the therapeutic agent, and such tablets can be employed in any of the methods (or used as) described herein. For example, where venetoclax is administered in combination with a RARA agonist (e.g., tamibarotene) and an HMA (e.g., azacitidine) to treat a hematological cancer (e.g., CLL or SLL), the venetoclax can be dosed according to a weekly ramp-up schedule over a period of weeks (e.g., five weeks) to the recommended daily dose of 400 mg. Where a method (or use) described herein includes administration of obinutuzumab, in addition to venetoclax, a RARA agonist (e.g., tamibarotene) and an HMA (e.g., azacitidine), the obinutuzumab can be administered intravenously at 100 mg on day 1; at 900 mg on day 2; and at 1,000 mg on days 8 and 15 prior to a ramp up dosing schedule with venetoclax. Modified versions of this treatment regimen are known in the art for subsequent cycles of treatment (e.g., cycle 2, cycles 3-6, and cycles 7-12).

In some embodiments, including the first, second, third, fourth, and fifth specific embodiments of the invention, determining whether cancer cells in a biological sample obtained from a patient have an MES above a requisite threshold level is achieved by first calculating the MES for each population of cells (a “sample”). This is done by applying a model that predicts monocytic status to each sample. The model is learned through an application of a machine learning algorithm (any reasonable machine learning algorithm capable of distinguishing between two classes will work, for example, an L1 regularized logistic regression, or logistic Lasso) to a whole genome readout of transcriptional or epigenetic activity (the “measurements”; for example, gene expression on multiple genes from RNA-seq) or a subset thereof (for example a 9-gene panel) for a number of known monocytic samples and a number of known primitive samples (for example, a collection of gene expression measurements on AML samples of known FAB, classifying FAB 0, 1, and 2 as primitive and FAB 4 and 5 as monocytic). The machine learning algorithm then predicts weights or rules to assign to each of the individual measurements. The model represents a combination of weights or rules that combines the measurements together into a single number (for example, a probability between 0 and 1) for each sample (referred to as the “MES score”). The model can be applied to samples for which the primitive or monocytic status is unknown. A determination of which samples have a score above a requisite threshold level is achieved by comparing the scores in the tested sample to the corresponding scores in a population of samples, wherein each of the samples is obtained from a different source (i.e. a different subject, a different cell line, a different xenograft). In some aspects of these embodiments, only primary tumor cell samples from subjects are used to determine the threshold level. In some aspects of these embodiments, at least some of the samples in the population will have been tested for responsiveness to a specific RARA agonist in order to establish: a) the lowest MES score of a sample in the population that responds to that specific RARA agonist (“lowest responder”); and, optionally, b) the highest MES score of a sample in the population that does not respond to that specific RARA agonist (“highest non-responder”). In these embodiments, a cutoff of MES score above which a test cell would be considered responsive to that specific RARA agonist is set to the number between the lowest responder and the highest non-responder that best separates the population of samples into responder and non-responder, taking into account desirable sensitivity and specificity constraints (for example setting a specificity of 90% to enable a high likelihood of a sample above the cutoff would be responsive, or a sensitivity of 90% to enable a high likelihood that any responsive sample will be above the cutoff).

### EXAMPLES

The studies described below were designed to investigate the features of insensitivity to, or resistance to, venetoclax in patients suffering from AML, and we have found that newly diagnosed, unfit AML patients with elevated RARA gene expression are enriched for features associated with primary resistance to venetoclax and clinical response to tamibarotene-plus-azacitidine. Venetoclax, in combination with azacitidine, is set to become a standard of care for many AML patients. While this treatment has seen good efficacy, some patients do not respond. Characterizing the patients who do not respond helps to identify therapies that could treat their disease. Tamibarotene, which is one of the RARA agonists we propose administering to the patients identified as described herein (e.g., patients diagnosed with the M4 or M5 subtype of AML or patients having MDS), is currently in clinical trials for AML patients with elevated expression of the RARA transcript.

The Beat AML consortium has generated RNA-seq data for 342 AML patients (Tyner et al., Nature 562:526-531, 2018). We removed the RNA-seq samples with less than 40% blasts (immature blood cells) for downstream analysis. Some patients had samples collected on multiple dates for RNA-seq analysis, and in that event, we analyzed only the earliest sample obtained per patient. Additionally, we retained only genes with a CPM (counts per million reads mapped) greater than one (1) for downstream analysis. RNA-seq count data were normalized using the function varianceStabilizingTransformation from the DESeq2 R package. The Cancer Genome Atlas (TCGA) has analyzed 200 AML primary patient samples (project code LAML), and we analyzed the RNA-seq data for 145 of those samples for protein-coding genes. RNA-seq count data were normalized using the function varianceStabilizingTransformation from the DESeq2 R package. We combined the normalized expression data for both datasets and normalized them together using quantile normalization.

Development of a monocytic expression signature in TCGA: We used the quantile normalized expression data of the TCGA samples with FAB (a French-American-British classification system) status M0, M1, M2, M4, and M5 to develop a monocytic expression signature (MES; the FAB classification being an advantage of the TCGA database). We selected ten genes known as markers of monocytic or primitive AML cell fate—CD14, CLEC7A (CD369), CD86, CD68, LYZ, MAFB, CD34, ITGAM (CD11b), FCGR1A (CD64), and KIT (CD117)—to develop a signature and used the expression of these genes to predict FAB M4/M5 vs. M0/M1/M2 status in the TCGA dataset by a logistic regression model with lasso regularization using the R package glmnet. This was done in a 10-fold cross validation manner and the largest lambda value within 1 standard error of the minimum was used for downstream predictions. Predictions were then made for all samples to generate a score for the signature. We designated samples with scores>0.5 as monocytic and designated samples with scores<0.5 as primitive.

Evaluation of gene expression features in BeatAML: We evaluated the monocytic expression signature in the BeatAML dataset using the quantile normalized expression and predictions made using the logistic regression model developed in the TCGA dataset. All other expression features used the expression normalized by variance stabilizing transformation. Samples with RARA expression greater than the 70% percentile of expression (i.e. in the top 30% of expression) were called RARA-high; other samples were called RARA-low.

Venetoclax sensitivity in BeatAML: The BeatAML dataset a/so included ex vivo sensitivity analysis for a panel of 121 inhibitors, including venetoclax, in a proliferation assay on 248 of the AML samples measured by area under the curve (AUC) and IC50. Only some of the patient samples were queried for each inhibitor. Of the samples with at least 40% blasts, 90 were tested for venetoclax ex vivo sensitivity. To define the best possible association of gene expression with venetoclax AUC, we performed a logistic regression model with lasso regularization to predict venetoclax AUC from the expression of all expressed genes using the expression matrix quantile normalized with the TCGA dataset. The R package glmnet was used as described above in the section “Development of monocytic expression signature in TCGA.”

To define samples sensitive to venetoclax, we first identified an IC50 that could separate sensitive samples from insensitive samples, as follows. We consulted two references (Bogenberger et al., Oncotarget 8:107206-107222, 2017) and Ramsey et al. Cancer Discovery 8:1566-1581, 2018) that defined sensitive AML cells in the range of 20-60 nM and resistant AML cells around 1 μM, and we choose 100 nM as a threshold that splits this range. We then mapped 100 nM IC50 values from the BeatAML venetoclax ex vivo assay to AUC, which is a more robust quantitative measure of sensitivity, by selecting the AUC below which all samples have an IC50<100 nM, which led to the AUC threshold of 130. At this threshold, we found 35 AML samples sensitive to venetoclax and 55 AML samples resistant to venetoclax.

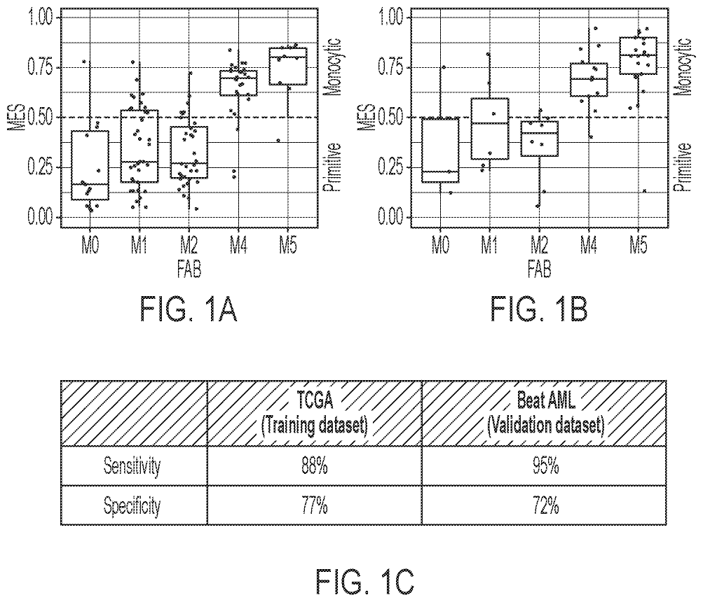

Results: In keeping with the methods described above, we developed a monocytic expression signature using the TCGA AML dataset to predict FAB M4/5 from FAB M0/1/2 using a set of ten (10) established markers of monocytic or primitive AML. When applied to the TCGA samples, the monocytic expression signature showed that the samples presented with a range of values from primitive to monocytic phenotype. As expected, the expression of the genes in the signature were associated with either the more monocytic or primitive samples depending on whether the genes were markers of primitive AML (KIT, CD34) or monocytic AML (FCGR1A, LYZ, CD68, ITGAM, CD86, MAFB, CD14, CLEC7A). Additionally, the signature score was strongly associated with FAB status, showing that it can accurately define FAB status (FIG. 1A). The monocytic expression signature was well correlated with the expression of RARA (Spearman's rho=0.6), and RARA-high samples were also more likely to be monocytic than RARA-low samples (81% of RARA-high samples are monocytic vs. 29% of RARA-low samples).

For validation, we then applied the monocytic expression signature to the BeatAML samples (an independent dataset), and the expression of the genes from the signature associated with the signature as expected and the expression signature predicted FAB with a similar level of accuracy as in the TCGA dataset (FIGS. 1B and 1C), indicating that the expression signature can accurately predict primitive vs monocytic AML status in datasets beyond the TCGA data. The signature was also correlated with RARA expression (Spearman's rho=0.58). RARA-high samples were more likely to be monocytic than RARA-low samples (77% of the RARA-high samples were monocytic vs. 37% of the RARA-low samples). This shows that RARA expression is associated with monocytic AML and suggests the RARA-high biomarker is selecting for monocytic AML.

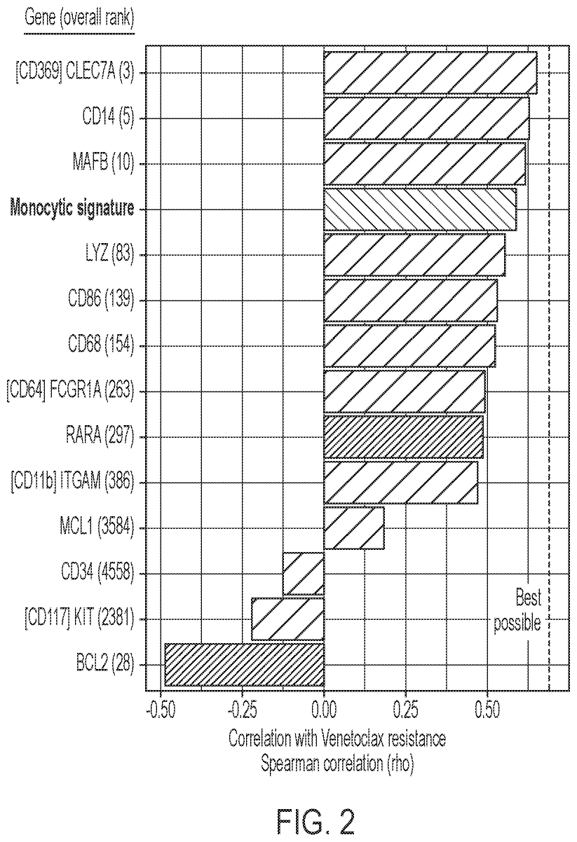

For those BeatAML samples with venetoclax ex vivo sensitivity data, we examined the relationship between venetoclax sensitivity and gene expression features (FIGS. 2 and 3). The monocytic expression signature as well as the individual gene markers of monocytic AML (CLEC7A/CD369, CD14, MAFB, LYZ, CD86, CD68, FCGR1A/CD64, ITGAM/CD11b) are all positively correlated with venetoclax AUC, indicating that monocytic AML is less sensitive to venetoclax. The primitive AML markers CD34 and KIT/CD117 are weakly negatively correlated with venetoclax AUC. RARA expression is positively correlated with venetoclax AUC, suggesting that the RARA-high biomarker may be selecting for AML insensitive to venetoclax. Amongst apoptotic regulators, BCL2 is negatively correlated with venetoclax AUC, and MCL1 is positively correlated with venetoclax AUC.

Evaluating gene expression features versus binary venetoclax sensitivity demonstrates that RARA expression and monocytic AML are both less sensitive to venetoclax (see the Table below). Amongst the 90 AML patients evaluated here for venetoclax sensitivity and gene expression, 35 (39%) were sensitive to venetoclax. Of the 21 RARA-high patients, none were sensitive to venetoclax compared to 51% of the RARA-low patients. Of the 44 monocytic AML patients, five (11%) were sensitive to venetoclax, and 65% of the primitive patients were sensitive. This suggests that both RARA expression and monocytic features (e.g., an MES) could be biomarkers for venetoclax insensitivity. However, the methods of treatment described herein can commence and can be carried out fully without assessment of RARA biomarker status.

Expression features of AML samples by ex vivo sensitivity to venetoclax

| | N sensitive | N total | Percent sensitive |
|---|---|---|---|
| All patients | 35 | 90 | 39% |
| RARA-high | 0 | 21 | 0% |
| RARA-low | 35 | 69 | 51% |
| Monocytic | 5 | 44 | 11% |
| Primitive | 30 | 46 | 65% |

In sum, in the BeatAML dataset, AML patient samples were evaluated for ex vivo sensitivity to venetoclax. This allowed us to determine which expression features were most predictive of sensitivity to venetoclax. RARA expression and a monocytic expression signature (MES), which are concordant themselves, were both highly enriched for insensitivity to venetoclax, implying that monocytic AML has high RARA expression and intrinsic insensitivity to venetoclax. This supports work from other groups showing more monocytic AML is less sensitive to venetoclax; primary resistance to venetoclax is associated with monocytic features in AML (Zhang, Blood, 2018; Pei et al. 2020, Kuusanmaki et al. 2020), and low-level monocytic clones present at diagnosis expand at relapse on treatment with venetoclax-plus-azacitidine (Pei et al., Cancer Discovery, 2018).

The studies set out above can be further described and summarized in the context of patient treatment as follows.

Super enhancer (SE) mapping in non-APL AML patient blasts identified RARa (also referenced herein as RARA) as a novel therapeutic target in approximately 30% of patients, who have elevated RARA gene expression. It was observed that the enhancer profile of this novel patient segment, where RARA expression was elevated, overlapped with the profile of mature monocytes (McKeown et al., Cancer Discovery, 7(10):1136-1153, 2017). Recently, several reports described AML with monocytic features associated with resistance to venetoclax (Ven), a BCL2 inhibitor that has emerged as a standard of care for treatment of patients with newly diagnosed (ND) unfit AML in combination with hypomethylating agents (HMAs) (Zhang, 2018; Kuusanmäki, 2019; Pei, 2020). Approximately one-third of patients do not respond to Ven plus HMAs including azacitidine (Aza) (DiNardo et al., Blood, 133(1):3-4, 2019; DiNardo et al., N. Engl. J. Med. 383:617-629, 2020), highlighting a continuing significant unmet need in ND unfit AML. Tamibarotene, a potent and selective RARa agonist, is in development for non-APL AML in combination with azacitidine and has demonstrated clinical activity with high rates of complete remission (CR) and deep CRs in RARA-positive (RARA+) ND unfit AML (DeBotton, 2019). Based on the overlap of monocytic features with RARA gene expression, we evaluated clinical samples of patients treated with tamibarotene plus azacitidine to correlate features of venetoclax resistance with the RARA biomarker and with clinical response to tamibarotene-plus-azacitidine.

In the methods, RARA gene expression in non-APL AML was evaluated in the TCGA and Beat AML RNA-seq datasets. AUC of cell viability curves were used to evaluate ex vivo sensitivity to compounds, including venetoclax, in the Beat AML dataset. A monocytic MES was developed using the expression of monocytic and primitive RNA markers in the TCGA dataset to analyze the monocytic phenotype. The MES used a logistic regression model with lasso regularization to distinguish FAB M4/5 (monocytic) from FAB M0/1/2 (primitive) using 10-fold cross-validation with 85% sensitivity and 80% specificity. The MES was then applied to the RNA-seq datasets from Beat AML and AML blasts from ND unfit AML patients in the ongoing tamibarotene-plus-azacitidine trial (NCT02807558), in which RARA-positive patients were determined by an RT-qPCR-based biomarker clinical trial assay (CTA). The MES, RARA expression, and venetoclax resistance-associated features were compared using Spearman's rho correlation; the association of the MES with the RARA biomarker and with IWG clinical responses in tamibarotene plus Aza treated patients was evaluated.

As noted, analysis of RNA-seq in TCGA non-APL AML pts demonstrated higher RARA expression in monocytic AML (FAB M4/M5) than primitive AML (FAB M0/M1/M2) (p<10⁻⁷, t-test). TCGA and Beat AML datasets also demonstrated that RARA expression was associated with the MES (rho=0.6 and 0.58), with approximately 80% of RARA-high patients across both databases having a high MES.

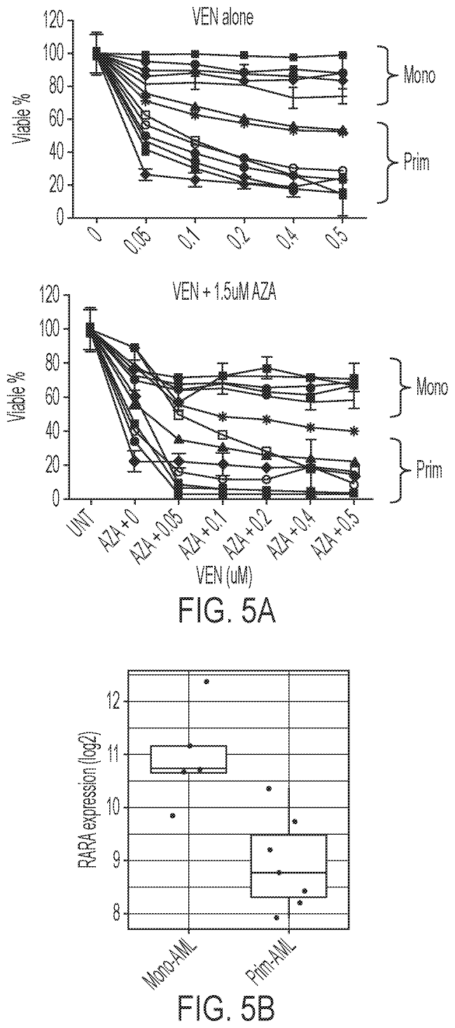

As further noted, we also elucidated the relationships of RARA expression, AML monocytic phenotypes, and venetoclax resistance. Of 121 inhibitors tested ex vivo in primary Beat AML patient samples, venetoclax was the inhibitor most associated with treatment resistance in RARA-positive vs. RARA-negative samples. Additionally, MES (rho=0.58), RARA (rho=0.48) and BCL2 expression (rho=−0.49) had similar magnitude of association with ex vivo venetoclax resistance, with RARA-positive samples showing much lower ex vivo sensitivity to venetoclax than RARA-negative samples (p=3×10⁻⁸). In 12 AML patient samples (Pei, 2020) treated with venetoclax±azacitidine ex vivo, RARA expression was higher in the monocytic leukemia stem cells resistant to Ven±Aza (p=0.005) (FIGS. 5A and 5B).

To evaluate whether the RARA-positive ND unfit AML patients in the ongoing tamibarotene-plus-azacitidine clinical trial were enriched for the monocytic phenotype of venetoclax resistance, RNA-seq was performed on enrolled patient AML blasts. Among 51 treated patients, 86% (19/22) of RARA-positive and 83% (24/29) of RARA-negative patients yielded RNA-seq results. RARA-positive patients were more monocytic than RARA-negative patients, as demonstrated by higher MES (p=7×10⁻⁵), with higher MCL1 (p=0.001), and lower BCL2, CD34, and CD117 expression (p=0.03, 8×10⁻⁶, 2×10⁻⁴, respectively). In patients with the best IWG response of CR/CRi, RARA+ pts (n=10) had higher MES than RARA-negative patients (n=9) (p=1.2×10⁻⁵).

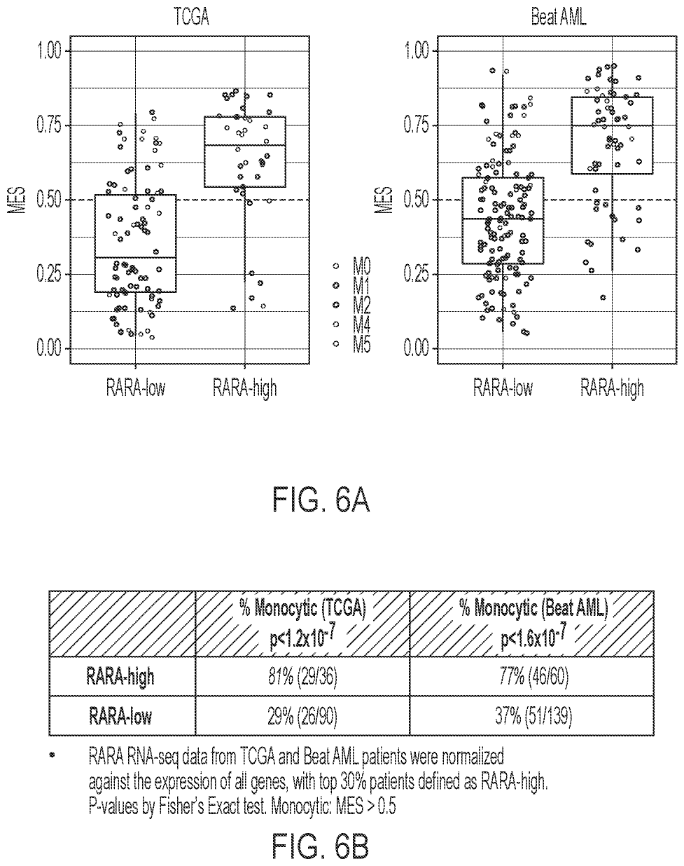

MES is higher in AML blasts with high RARA expression, and both RARA expression and MES are associated with resistance to venetoclax ex vivo: As shown in FIGS. 6A and 6B, high RARA expression identifies an AML patient population enriched for high monocytic gene expression in TCGA and Beat AML databases. RARA RNA-seq data from TCGA and Beat AML patients were normalized against the expression of all genes, with the top 30% of patients defined as RARA-high. P-values by Fisher Exact test. Monocytic MES>0.5.

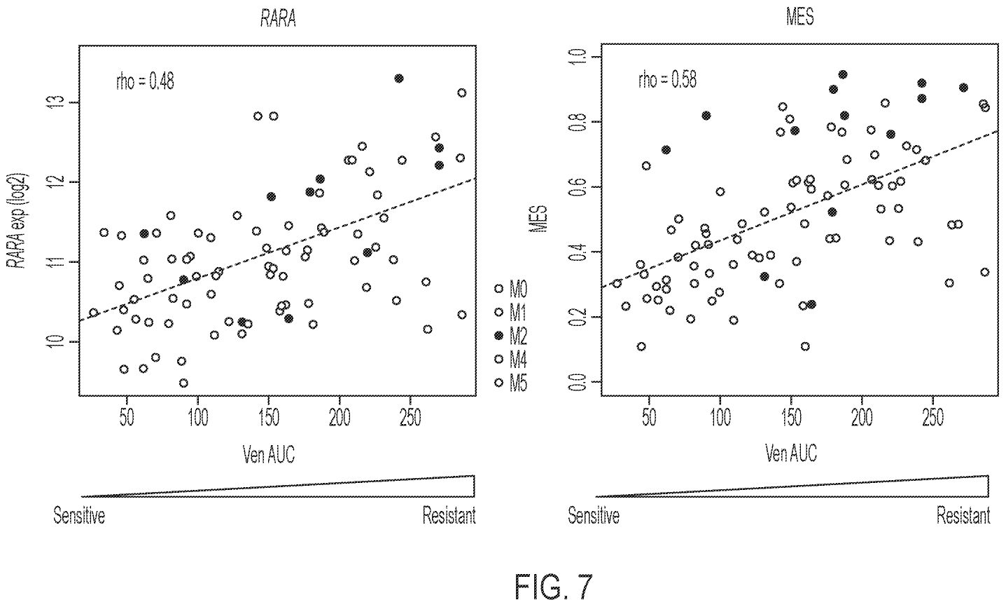

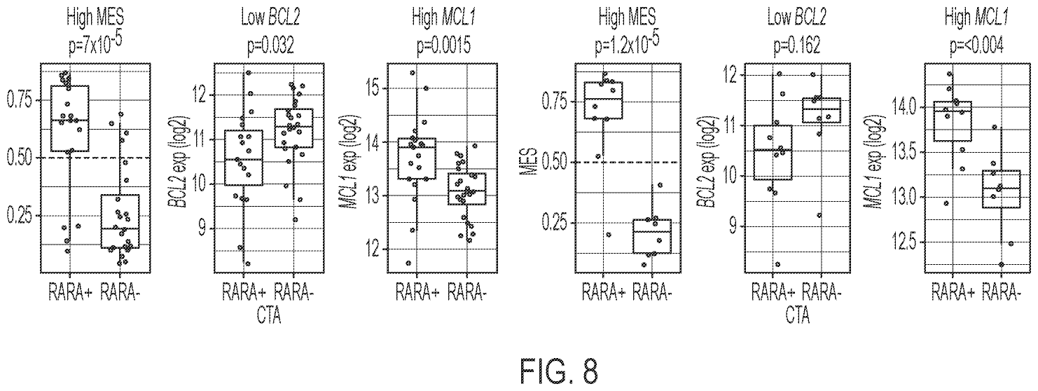

In primary AML cultures, RARA expression and MES are associated with resistance to venetoclax (see FIG. 7; Spearman correlation (rho) of normalized RARA expression (left) or MES (right) vs. venetoclax response across 90 AML primary cultures (Beat AML)). RARA positive ND unfit AML patients, including those with clinical response to tamibarotene-plus-azacitidine, are enriched for features associated with resistance to venetoclax: Forty-three ND, unfit AML patients enrolled in a clinical trial for tamibarotene had RNA-seq data from blasts isolated with the RARA biomarker clinical trial assay (CTA). 80% (15/19) of RARA-positive patients are classified as monocytic by MES (MES>0.5), and 17% (4/24) of RARA-negative patients are classified as monocytic by MES (MES>0.5) (see FIG. 8). These results support our conclusions that, in ND unfit AML, RARA-positive patients, including those with clinical responses to tamibarotene-plus-azacitidine, are enriched for monocytic features associated with resistance to venetoclax. Approximately 80% of RARA-positive ND unfit AML patients in our clinical studies have a monocytic phenotype associated with resistance to venetoclas, which includes lower BCL2 and higher MCL1 expression. Thus, we propose tamibarotene, alone or in combination with azacitidine, as a targeted regimen for the treatment of ND unfit AML. As this genomically defined subset of AML patients may be resistant to upfront SOC therapy with venetoclax, the methods described herein provide treatment options for patients who are less likely to respond to venetoclax-plus-azacitidine and for whom a high unmet need remains.

1. Use of a therapeutically effective amount of tamibarotene or a pharmaceutically acceptable salt thereof in treating a patient who has been diagnosed with acute myelomonocytic leukemia (the M4 subtype of acute myeloid leukemia (AML)) or acute monocytic leukemia (the M5 subtype of AML).

2-50. (canceled)
